(19) United States
(12) Patent Application Publication
(10) Pub. No.: US 2024/0350398 A1
(43) Pub. Date: Oct. 24, 2024

(54) PURIFIED PRODUCT OF CITRUS OIL AND PRODUCTION METHOD OF PURIFIED PRODUCT OF CITRUS OIL

(71) Applicant: TAKASAGO INTERNATIONAL CORPORATION, Tokyo (JP)

(72) Inventors: Naoto TERADA, Kanagawa (JP); Tadahiro HIRAMOTO, Kanagawa (JP)

(73) Assignee: TAKASAGO INTERNATIONAL CORPORATION, Tokyo (JP)

(21) Appl. No.: 18/701,121

(22) PCT Filed: Oct. 5, 2022
(86) PCT No.: PCT/JP2022/037309

§ 371 Date: Apr. 12, 2024

(30) Foreign Application Priority Data
Oct. 13, 2021 (JP) 2021-168177

(51) Int. Cl. A61K 8/92; A23L 2/56; A23L 5/20; A23L 27/10; A23L 27/12; A61K 36/752; A61Q 11/00; A61Q 19/00; C11B 3/00; C11B 3/06; C11B 3/10; C11B 3/12

(52) U.S. Cl. CPC A61K 8/922; A23L 2/56; A23L 5/273; A23L 27/11; A23L 27/115; A23L 27/13; A61K 36/752; A61Q 11/00; A61Q 19/00; C11B 3/001; C11B 3/06; C11B 3/10; C11B 3/12; A61K 2800/10

(57) ABSTRACT

The present invention relates to a purified product of a citrus oil, in which an oil derived from a citrus peol is used as a raw material, a content of linoleic acid is 0.015 parts by mass or less with respect to 1 part by mass of citral, or 0.063 parts by mass or less with respect to 1 part by mass of linalool, or 0.083 parts by mass or less with respect to 1 part by mass of decanal, and a content of sesquiterpene hydrocarbons is 20 mass % or less.

### TECHNICAL FIELD

The present invention relates to a purified product of a citrus oil and a production method of the purified product of a citrus oil.

### BACKGROUND ART

An oil derived from a peel of a citrus such as lemon, lime, orange, or grapefruit may be used for a beverage or food product, a fragrance or cosmetic product, a pharmaceutical product, or an oral care product, as a material that imparts citrus aroma as it is or in addition to being blended into a fragrance or flavor. In addition, the oil contains a large amount of terpene hydrocarbons (mainly monoterpene hydrocarbons) which are oxidized and degraded over time by light or heat in the presence of oxygen, water or an acid to produce an abnormal-odor substance. Therefore, by reducing the terpene hydrocarbons by rectification, organic solvent extraction, and the like, generation of an abnormal-odor substance may be suppressed, and the oil may be used, as it is or in addition to being blended into a fragrance or flavor, for a beverage or food product, a fragrance or cosmetic product, a pharmaceutical product, an oral care product, or the like after concentrating oxygenated compounds such as alcohols and aldehydes having a high degree of contribution to aroma.

On the other hand, it is known that when a beverage or food product containing an oil derived from a citrus peel, a concentrate obtained by reducing terpene hydrocarbons from the oil, or a fragrance or flavor containing the oil and the concentrate is exposed to light, an abnormal odor is generated and palatability is reduced. It is known that generation of an abnormal odor involves plant wax (Patent Literature 1) and citral (Patent Literature 2) in addition to the terpene hydrocarbons, all of which are contained in an oil derived from a citrus peel, a concentrate obtained by reducing terpene hydrocarbons from the oil, or a fragrance or flavor containing the oil and the concentrate, and such substances are oxidized or hydrolyzed, whereby an abnormal-odor component is generated. For the purpose of suppressing occurrence of the abnormal odor, a method of removing chlorophyll, which is considered to be a factor that promotes oxidation of terpene hydrocarbons, by a treatment with activated carbon (Patent Literature 3), a method of removing plant wax by a treatment with an adsorbent such as activated carbon (Patent Literature 1), and the like have been reported. In addition, it has been reported that a purified product of a citrus essential oil obtained by subjecting, to a rectification treatment, a distillate obtained by subjecting an oil derived from a citrus peel to a thin-film distillation treatment hardly generates an abnormal odor (Patent Literature 4).

In addition, there is no description of an oil derived from a citrus peel, a concentrate obtained by reducing terpene hydrocarbons from the oil, or a fragrance or flavor containing the oil and the concentrate, but it has been reported that when a beverage containing an unsaturated fatty acid derived from a fruit juice or a fragrance or flavor is exposed to light, a component causing an abnormal odor is generated from the unsaturated fatty acid (Patent Literature 5). The oil derived from a citrus peel contains a certain amount of unsaturated fatty acid, but there has been no report on a technique for reducing the unsaturated fatty acid contained in the oil derived from a citrus peel or the concentrate obtained by reducing terpene hydrocarbons from the oil for the purpose of suppressing generation of the component causing an abnormal odor.

### CITATION LIST

### Patent Literature

Patent Literature 1: JP2005-143370A

Patent Literature 2: JP2017-086013A

Patent Literature 3: US2010/0189872

Patent Literature 4: WO2016/121186

Patent Literature 5: JP2018-099092A

### SUMMARY OF INVENTION

### Technical Problem

On the other hand, according to the study of the present inventors, there is a problem that an effect of suppressing an abnormal odor obtained by the method described above is limited, or even when the effect of suppressing an abnormal odor is obtained, components contributing to an aroma of the oil derived from a citrus peel and the fragrance or flavor containing the oil are partially lost, or component balance is largely lost by a treatment, and the aroma originally possessed by the oil derived from a citrus peel and the fragrance or flavor containing the oil is impaired.

The present invention has been made in view of the above circumstances of the related art, and an object thereof is to provide a purified product of a citrus oil capable of suppressing, when being used in a beverage or food product, occurrence of an abnormal odor even when the beverage or food product is exposed to light, and capable of imparting an aroma originally possessed by an oil derived from a citrus peel.

### Solution to Problem

As a result of intensive studies to solve the above problems, the present inventors have found that a substance generated by oxidation of linoleic acid, which is a kind of unsaturated fatty acid, greatly contributes to generation of an abnormal odor when a beverage or food product containing an oil derived from a citrus peel, a concentrate obtained by reducing terpene hydrocarbons from the oil, or a fragrance or flavor containing the oil and the concentrate is exposed to light. The present inventors further have found that occurrence of an abnormal odor can be suppressed to a perceived level or less by setting a content of linoleic acid with respect to a content of a specific main aroma component in a purified product of a citrus oil to a specific value or less. At the same time, the present inventors have found that an aroma originally possessed by the oil derived from a citrus peel is favorably imparted by setting a content of sesquiterpene hydrocarbons in the purified product of a citrus oil to a specific value or less, and have completed the present invention.

That is, the present invention relates to the following <1> to <10>.
<1> A purified product of a citrus oil, in which an oil derived from a citrus peel is used as a raw material,
a content of linoleic acid is 0.015 parts by mass or less with respect to 1 part by mass of citral, or 0.063 parts by mass or less with respect to 1 part by mass of linalool, or 0.083 parts by mass or less with respect to 1 part by mass of decanal, and
a content of sesquiterpene hydrocarbons is 20 mass % or less.
<2> The purified product of a citrus oil according to <1>, in which an oil derived from a lemon peel is used as a raw material,
a content of linoleic acid is 0.015 parts by mass or less with respect to 1 part by mass of citral, and
a content of sesquiterpene hydrocarbons is 20 mass % or less.
<3> The purified product of a citrus oil according to <1>, in which an oil derived from a lime peel is used as a raw material,
a content of linoleic acid is 0.008 parts by mass or less with respect to 1 part by mass of citral, and
a content of sesquiterpene hydrocarbons is 20 mass % or less.
<4> The purified product of a citrus oil according to <1>, in which an oil derived from an orange peel is used as a raw material,
a content of linoleic acid is 0.063 parts by mass or less with respect to 1 part by mass of linalool, and
a content of sesquiterpene hydrocarbons is 10 mass % or less.
<5> The purified product of a citrus oil according to <1>, in which an oil derived from a grapefruit peel is used as a raw material,
a content of linoleic acid is 0.083 parts by mass or less with respect to 1 part by mass of decanal, and
a content of sesquiterpene hydrocarbons is 10 mass % or less.
<A fragrance or flavor composition, comprising the purified product of a citrus oil according to any of <1>to <5>.
<7> A beverage or food product, a fragrance or cosmetic product, a pharmaceutical product, or an oral care product, comprising the fragrance or flavor composition according to <6>.
<8> A production method of the purified product of a citrus oil according to any of <1> to <5>, including at least one step selected from the group consisting of a step of subjecting an oil as a raw material to an aqueous alkali solution washing treatment, a step of subjecting an oil as a raw material to an ion exchange resin treatment, and a step of subjecting an oil as a raw material to a treatment in which thin-film distillation and liquid-liquid extraction are combined.
<9> A production method of the purified product of a citrus oil according to any of <1> to <5>, including:
a step of subjecting an oil as a raw material to an aqueous alkali solution washing treatment, in which
an alkali substance used in the aqueous alkali solution washing treatment is a strong base, and
when the oil as the raw material is treated without being concentrated and is subjected to the aqueous alkali solution washing treatment, an amount of the alkali substance used is 0.05 mol or less with respect to 1 kg of a treatment target.
<10> A production method of the purified product of a citrus oil according to any of <1> to <5>, including:
a step of subjecting an oil as a raw material to an ion exchange resin treatment, in which
an ion exchange resin used in the ion exchange resin treatment is an anion exchange resin, and
an amount of the anion exchange resin used is 0.03 kg or more with respect to 1 L of a treatment target.

### Advantageous Effects of Invention

The purified product of a citrus oil according to the present invention can suppress,

when being used for a beverage or food product, occurrence of an abnormal odor even when the beverage or food product is exposed to light, and can impart an aroma originally possessed by the oil derived from a citrus peel.

### DESCRIPTION OF EMBODIMENTS

Hereinafter, the present invention will be described in detail, but the following contents are merely an example of preferred embodiments, and the present invention is not limited thereto.

### Purified Product of Citrus Oil

A purified product of a citrus oil according to the present invention contains an oil derived from a peel of a citrus as a raw material. The oil derived from a peel of a citrus is an oil (cold pressed oil) which contains linoleic acid and is obtained by compressing the peel of a citrus or by extracting the peel of a citrus with an organic solvent or the like and then removing the solvent by distillation or the like.

Examples of the citrus include lemon, lime, orange (suite orange, sour orange, mandarin orange), and grapefruit.

In the purified product of a citrus oil according to the present invention, a content of linoleic acid is 0.015 parts by mass or less with respect to 1 part by mass of citral, or 0.063 parts by mass or less with respect to 1 part by mass of linalool, or 0.083 parts by mass or less with respect to 1 part by mass of decanal, and a content of sesquiterpene hydrocarbons is 20 mass % or less.

When lemon is used as the citrus, a specific main aroma component in the purified product of a citrus oil according to the present invention is citral.

In the case where lemon is used as the citrus, when the content of the linoleic acid with respect to 1 part by mass of citral in the purified product of a citrus oil according to the present invention is 0.015 parts by mass or less and the content of the sesquiterpene hydrocarbons is in a range of 20 mass % or less, occurrence of an abnormal odor can be suppressed at the time of using the purified product of a citrus oil according to the present invention for a beverage or food product or the like and exposing the beverage or food product to light, and an aroma originally processed by the oil derived from a citrus peel can be imparted.

The content of linoleic acid with respect to 1 part by mass of citral in the case where lemon is used as the citrus is preferably 0.015 parts by mass or less, more preferably 0.01 parts by mass or less, and still more preferably 0.008 parts by mass or less, from the viewpoint of suppressing occurrence of an abnormal odor.

In a case where lime is used as the citrus, a specific main aroma component in the purified product of a citrus oil according to the present invention is citral.

In the case where lime is used as the citrus, when the content of the linoleic acid with respect to 1 part by mass of citral in the purified product of a citrus oil according to the present invention is 0.008 parts by mass or less and the content of the sesquiterpene hydrocarbons is in a range of 20 mass % or less, occurrence of an abnormal odor can be suppressed at the time of using the purified product of a citrus oil according to the present invention for a beverage or food product or the like and exposing the beverage or food product to light, and an aroma originally processed by the oil derived from a citrus peel can be imparted.

The content of the linoleic acid with respect to 1 part by mass of citral in the case where lime is used as the citrus is preferably 0.008 parts by mass or less, more preferably 0.005 parts by mass or less, and still more preferably 0.004 parts by mass or less, from the viewpoint of suppressing occurrence of an abnormal odor.

In a case where orange is used as the citrus, a specific main aroma component in the purified product of a citrus oil according to the present invention is linalool.

In the case where orange is used as the citrus, when the content of the linoleic acid with respect to 1 part by mass of linalool in the purified product of a citrus oil according to the present invention is 0.063 parts by mass or less and the content of the sesquiterpene hydrocarbons is in a range of 10 mass % or less, occurrence of an abnormal odor can be suppressed at the time of using the purified product of a citrus oil according to the present invention for a beverage or food product or the like and exposing the beverage or food product to light, and an aroma originally processed by the oil derived from a citrus peel can be imparted.

The content of the linoleic acid with respect to 1 part by mass of linalool in the case where orange is used as the citrus is preferably 0.063 parts by mass or less, more preferably 0.042 parts by mass or less, and still more preferably 0.032 parts by mass or less, from the viewpoint of suppressing occurrence of an abnormal odor.

In a case where grapefruit is used as the citrus, a specific main aroma component in the purified product of a citrus oil according to the present invention is decanal.

In the case where grapefruit is used as the citrus, when the content of the linoleic acid with respect to 1 part by mass of decanal in the purified product of a citrus oil according to the present invention is 0.083 parts by mass or less and the content of the sesquiterpene hydrocarbons is in a range of 10 mass % or less, occurrence of an abnormal odor can be suppressed at the time of using the purified product of a citrus oil according to the present invention for a beverage or food product or the like and exposing the beverage or food product to light, and an aroma originally processed by the oil derived from a citrus peel can be imparted.

The content of the linoleic acid with respect to 1 part by mass of decanal in the case where grapefruit is used as the citrus is preferably 0.083 parts by mass or less, more preferably 0.055 parts by mass or less, and still more preferably 0.042 parts by mass or less, from the viewpoint of suppressing occurrence of an abnormal odor.

In a case where lemon or lime is used as the citrus, the content of the sesquiterpene hydrocarbons in the purified product of a citrus oil according to the present invention is preferably 20 mass % or less, more preferably 15 mass % or less, and still more preferably 10 mass % or less.

In a case where orange or grapefruit is used as the citrus, the content of the sesquiterpene hydrocarbons in the purified product of a citrus oil according to the present invention is preferably 10 mass % or less, more preferably 7.5 mass % or less, and still more preferably 5 mass % or less.

When the content of the sesquiterpene hydrocarbons is out of the above range, an effect of the aroma derived from the sesquiterpene hydrocarbons on a fragrance or flavor note increases, and the aroma originally possessed by the oil derived from a citrus peel, which is the raw material of the purified product of a citrus oil according to the present invention, is impaired. On the other hand, when the content of the sesquiterpene hydrocarbons is within the above range, the aroma derived from the sesquiterpene hydrocarbons does not excessively affect the fragrance or flavor note, and therefore, the aroma originally possessed by the oil derived from a citrus peel, which is the raw material of the purified product of a citrus oil according to the present invention, can be satisfactorily maintained.

The sesquiterpene hydrocarbons are not particularly limited as long as being contained in the oil derived from a citrus peel, and examples thereof include β-caryophyllene, α-farnesene, β-farnesene, α-bergamotene, α-bisabolene, β-bisabolene, α-copaene, β-copaene, α-cubebene, β-cubebene, α-elemene, β-elemene, γ-elemene, δ-elemene, α-humulene, germacrene B, germacrene D, bicyclogermacrene, valencene, α-muurolene, δ-guaiene, γ-cadinene, δ-cadinene, α-santalene, β-santalene, and α-selinene.

The purified product of a citrus oil according to the present invention may contain, in addition to the components described above, other components as long as the effects of the present invention are not impaired.

The other components are not particularly limited, and examples thereof include oxygenated compounds and terpene hydrocarbons contained in the oil derived from a citrus peel as a raw material, and organic solvents used in a process of producing the purified product of a citrus oil.

### Production Method of Purified Product of Citrus Oil

A production method of the purified product of a citrus oil according to the present invention includes at least one step selected from the group consisting of a step of subjecting an oil as a raw material to an aqueous alkali solution washing treatment, a step of subjecting an oil as a raw material to an ion exchange resin treatment, and a step of subjecting an oil as a raw material to a treatment in which thin-film distillation and liquid-liquid extraction are combined.

In the present invention, the “oil as a raw material” means an oil derived from a citrus peel, or an oil obtained by concentrating oxygenated compounds by reducing terpene hydrocarbons from an oil derived from a citrus peel by a method such as rectification or organic solvent extraction.

### Aqueous Alkali Solution Washing Treatment

The step of performing an aqueous alkali solution washing treatment preferably includes
a step of bringing, by mixing or dropping, an oil as a raw material into contact with an aqueous solution in which an alkali substance is dissolved to distribute linoleic acid as salt in the aqueous solution,
a step of separating, by static separation or centrifugation, the oil and the aqueous solution in which linoleic acid is distributed as salt,
a step of bringing, by mixing or dropping, the separated oil into contact with an aqueous solution in which an acidic substance is dissolved to neutralize the alkali substance which remains in a trace amount, and
a step of separating the aqueous solution and the oil by static separation or centrifugation.

The step of performing the aqueous alkali solution washing treatment may include a step of dehydrating the obtained oil with diatomaceous earth, sodium sulfate, or the like.

The alkali substance used is not particularly limited, and examples thereof include weak bases such as sodium carbonate, potassium carbonate, ammonium carbonate, sodium bicarbonate, potassium bicarbonate, ammonium bicarbonate, ammonium carbamate, magnesium hydroxide, magnesium oxide, disodium hydrogen phosphate, dipotassium hydrogen phosphate, potassium pyrophosphate, sodium pyrophosphate, sodium polyphosphate, potassium polyphosphate, sodium metaphosphate, and potassium metaphosphate: and strong bases such as sodium hydroxide, potassium hydroxide, calcium hydroxide, calcium oxide, trisodium phosphate, tripotassium phosphate, and sodium methoxide. These alkali substances may be used alone or in combination of two or more thereof.

The acidic substance to be used is not particularly limited, and examples thereof include adipic acid, benzoic acid, citric acid, glucono-δ-lactone, gluconic acid, DL-tartaric acid. L-tartaric acid, carbon dioxide, lactic acid, glacial acetic acid, disodium dihydrogen pyrophosphate, fumaric acid, monosodium fumarate, DL-malic acid, phosphoric acid, potassium dihydrogen phosphate, sodium dihydrogen phosphate, L-ascorbic acid, hydrochloric acid, L-glutamic acid, cinnamic acid, succinic acid, oxalic acid, and ammonium sulfate. These acidic substances may be used alone or in combination of two or more thereof.

An amount of the alkali substance used is not particularly limited, and is preferably 0.01 mol or more with respect to 1 kg of a treatment target.

Only when a strong base is used and the oil as the raw material is treated without being concentrated, the amount of the alkali substance used is preferably 0.05 mol or less with respect to 1 kg of the treatment target. In a case where the amount of the alkali substance used exceeds 0.05 mol, a part of aroma components may decrease after the treatment, and the fragrance or flavor note derived from the raw material may not be maintained.

A treatment temperature in the aqueous alkali solution washing treatment is not particularly limited, and is preferably 0° C. to 100° C., and more preferably 5° C. to 60° C. For the purpose of improving the separability, an organic solvent or an inorganic

salt may be used in combination in the step of bringing, by mixing or dropping, an oil as a raw material into contact with an aqueous alkali solution in which an alkali substance is dissolved to distribute linoleic acid as salt in the aqueous solution, and/or in the step of bringing, by mixing or dropping. the separated oil into contact with an acidic aqueous solution in which an acidic substance is dissolved to neutralize the alkali substance which remains in a trace amount.

The organic solvent is not particularly limited, and examples thereof include methanol, ethanol, 1-propanol, 2-propanol, 1-butanol, 2-butanol, propylene glycol, glycerin, acetone, methyl acetate, ethyl acetate, hexane, heptane, edible oils and fats, and middle chain fatty acid triacylglycerol (MCT). These organic solvents may be used alone or in combination of two or more thereof.

The inorganic salt is not particularly limited, and examples thereof include sodium chloride, potassium chloride, magnesium chloride, sodium sulfate, potassium sulfate, and magnesium sulfate. These inorganic salts may be used alone or in combination of two or more thereof.

### Ion Exchange Resin Treatment

The step of performing an ion exchange resin treatment preferably includes
a step of bringing an oil as a raw material into contact with an anion exchange resin, thereby causing linoleic acid to be adsorbed on the anion exchange resin, and
a step of separating the anion exchange resin and the oil by filtration, centrifugation, or the like.

A step of separating the anion exchange resin and the oil as the raw material may be omitted by passing the oil through the anion exchange resin filled in advance in a column having an outlet structure in which an ion exchange resin does not leak. The step of performing the ion exchange resin treatment may further include a step of dehydrating the obtained oil with diatomaceous earth, sodium sulfate, or the like, as necessary.

The anion exchange resin is not particularly limited, and for example, a strongly basic anion exchange resin such as DIAION PA308, PA316, PA408, or PA418 (all manufactured by Mitsubishi Chemical Corporation), or a weakly basic anion exchange resin such as DIAION WA10, WA20, or WA30 (all manufactured by Mitsubishi Chemical Corporation) can be used. These anion exchange resins may be used alone or in combination of two or more thereof.

An amount of the anion exchange resin used is preferably 0.03 kg or more with respect to 1 L of the treatment target. In a case where the amount of the anion exchange resin used is less than 0.03 kg, the linoleic acid may not be sufficiently removed.

In addition, a cation exchange resin may be used in combination in order to remove a substance which is slightly desorbed from the anion exchange resin. The cation exchange resin is not particularly limited, and for example, a strongly acidic cation exchange resin such as DIAION PK216 and PK228 (manufactured by Mitsubishi Chemical Corporation), and a weakly acidic cation exchange resin such as DIAION WK10, WK11, or WK100 (manufactured by Mitsubishi Chemical Corporation) can be used. These cation exchange resins may be used alone or in combination of two or more thereof.

A treatment temperature in the ion exchange resin treatment is not particularly limited, and is preferably 0° C. to 100° C., and more preferably 5° C. to 60° C.

### Treatment in which Thin-Film Distillation and Liquid-Liquid Extraction are Combined

The step of performing a treatment in which thin-film distillation and liquid-liquid extraction are combined preferably includes
a step of subjecting an oil as a raw material to the thin-film distillation to recover a volatile fraction,
a step of bringing the volatile fraction into contact with a mixed liquid of water and an organic solvent to perform liquid-liquid extraction,
a step of separating an extract liquid and the oil after extraction by static separation, centrifugation, or the like, and
a step of distilling the extract liquid to remove the water and the organic solvent.

The step of performing a treatment in which thin-film distillation and liquid-liquid extraction are combined may further include a step of dehydrating the obtained oil with diatomaceous earth, sodium sulfate, or the like.

A thin-film distillation apparatus used in the thin-film distillation is not particularly limited, and examples thereof include a rotary film distillation apparatus, a falling film distillation apparatus, a centrifugal film distillation apparatus, a molecular distillation apparatus, and a short path distillation apparatus.

A treatment pressure of the thin-film distillation is not particularly limited, and is preferably 0.001 kPa to 2.0 kPa, and more preferably 0.01 kPa to 2.0 kPa.

A treatment temperature of the thin-film distillation is not particularly limited, and is preferably 10° C. to 130° C., and more preferably 10° C. to 100° C.

An operation of recovering the volatile fraction by thin-film distillation of a nonvolatile fraction after thin-film distillation may be repeated until a sufficient amount of the volatile fraction can be recovered.

The liquid-liquid extraction step and the step of separating the extract liquid and the oil after the extraction may be performed in a batch manner in which the volatile fraction of the oil as the raw material and the mixed liquid of water and the organic solvent are brought into contact with each other all at once, and then the extract liquid and the oil after the extraction are separated, or may be performed in a continuous manner in which the volatile fraction of the oil as the raw material and the mixed liquid of water and the organic solvent are partially and continuously supplied at a predetermined flow rate and brought into contact with each other, and the extract liquid and the oil after the extraction are partially and continuously separated.

The liquid-liquid extraction step and the step of separating the extract liquid and the oil after the extraction may be performed only by one treatment, or the step of bringing the oil after the extraction obtained after one treatment into contact with the mixed liquid of water and the organic solvent and then separating the extract liquid and the oil after the extraction may be repeated until sufficient component recovery is performed, so as to collect the additional extract liquid, which may be mixed with the extract liquid obtained after one treatment. Alternatively, a multistage treatment may be performed in which a step of separating a volatile fraction of an oil as a raw material and a mixed liquid of water and an organic solvent after countercurrent contact is continuously performed a plurality of times using a countercurrent distribution type continuous extraction apparatus, until sufficient component recovery is performed.

The apparatus used in the liquid-liquid extraction step and the step of separating the extract liquid and the oil after extraction is not particularly limited, and examples of a batch type apparatus include an extraction tank having a stirring blade or a circulation pump for stirring, and an apparatus in which a centrifuge is added to the extraction tank, and examples of a continuous type apparatus include a mixer-settler type extraction apparatus, a spray tower type extraction apparatus, a porous plate tower type extraction apparatus, and a countercurrent distribution type centrifugal extraction apparatus.

The organic solvent used for liquid-liquid extraction is not particularly limited, and methanol, ethanol, 1-propanol, 2-propanol, 1-butanol, 2-butanol, propylene glycol, glycerin, and acetone are preferable. These organic solvents may be used alone or in combination of two or more thereof.

An amount of the organic solvent used is not particularly limited, and is preferably 1 kg or more with respect to 1 kg of the volatile fraction of the oil as the raw material to be extracted.

A ratio of water to the organic solvent in the mixed liquid is not particularly limited, and is preferably 0.5 parts by mass or more of the organic solvent with respect to 1 part by mass of water.

A treatment temperature of liquid-liquid extraction is not particularly limited, and is preferably 0° C. to 100° C., and more preferably 5° C. to 60° C.

For the purpose of improving separability or the like, an organic solvent or an inorganic salt may be used in combination in the step of bringing the volatile fraction into contact with a mixed liquid of water and an organic solvent to perform liquid-liquid extraction, and/or in the step of separating the extract liquid and the oil after extraction by static separation, centrifugation, or the like.

The organic solvent is not particularly limited, and examples thereof include acetone, methyl acetate, ethyl acetate, hexane, heptane, edible fats and oils, and triacylglycerol (MCT). These organic solvents may be used alone or in combination of two or more thereof.

The inorganic salt is not particularly limited, and examples thereof include sodium chloride, potassium chloride, magnesium chloride, sodium sulfate, potassium sulfate, and magnesium sulfate. These inorganic salts may be used alone or in combination of two or more thereof.

In the step of bringing the volatile fraction into contact with a mixed liquid of water and an organic solvent to perform liquid-liquid extraction, and/or in the step of separating the extract liquid and the oil after extraction by static separation, centrifugation, or the like, an alkali substance or an acidic substance may be used in combination for the purpose of pH adjustment.

The alkali substance is not particularly limited, and examples thereof include sodium carbonate, potassium carbonate, ammonium carbonate, sodium bicarbonate, potassium bicarbonate, ammonium bicarbonate, ammonium carbamate, magnesium hydroxide, magnesium oxide, disodium hydrogen phosphate, dipotassium hydrogen phosphate, potassium pyrophosphate, sodium pyrophosphate, sodium polyphosphate, potassium polyphosphate, sodium metaphosphate, potassium metaphosphate, sodium hydroxide, potassium hydroxide, calcium hydroxide, calcium oxide, trisodium phosphate, tripotassium phosphate, and sodium methoxide. These alkali substances may be used alone or in combination of two or more thereof.

The acidic substance is not particularly limited, and examples thereof include adipic acid, benzoic acid, citric acid, glucono-δ-lactone, gluconic acid, DL-tartaric acid, L-tartaric acid, carbon dioxide, lactic acid, glacial acetic acid, disodium dihydrogen pyrophosphate, fumaric acid, monosodium fumarate, DL-malic acid, phosphoric acid, potassium dihydrogen phosphate, sodium dihydrogen phosphate, L-ascorbic acid, hydrochloric acid, L-glutamic acid, cinnamic acid, succinic acid, oxalic acid, and ammonium sulfate. These acidic substances may be used alone or in combination of two or more thereof.

The step of distilling the extract liquid to remove the water and the organic solvent may be performed by one distillation or may be performed by distillation for a plurality of times. When formation of a water layer containing an organic solvent is observed during the step, the water layer may be removed by static separation, centrifugation, or the like.

A distillation apparatus used in the step of distilling the extract liquid to remove the water and the organic solvent is not particularly limited, and a thin-film distillation apparatus, a simple distillation apparatus, and a rectification apparatus are preferable.

A distillation treatment pressure is not particularly limited, and is preferably 102 kPa or less.

A distillation treatment temperature is not particularly limited, and is preferably 10° C. to 130° C., and more preferably 10° C. to 100° C.

In the step of distilling the extract liquid to remove the water and the organic solvent, an inorganic salt may be used in combination for the purpose of promoting formation and separation of a water layer containing the organic solvent.

The inorganic salt is not particularly limited, and examples thereof include sodium chloride, potassium chloride, magnesium chloride, sodium sulfate, potassium sulfate, and magnesium sulfate. These inorganic salts may be used alone or in combination of two or more thereof.

In the step of distilling the extract liquid to remove the water and the organic solvent, an alkali substance or an acidic substance may be used in combination for the purpose of pH adjustment.

The alkali substance is not particularly limited, and examples thereof include sodium carbonate, potassium carbonate, ammonium carbonate, sodium bicarbonate, potassium bicarbonate, ammonium bicarbonate, ammonium carbamate, magnesium hydroxide, magnesium oxide, disodium hydrogen phosphate, dipotassium hydrogen phosphate, potassium pyrophosphate, sodium pyrophosphate, sodium polyphosphate, potassium polyphosphate, sodium metaphosphate, potassium metaphosphate, sodium hydroxide, potassium hydroxide, calcium hydroxide, calcium oxide, trisodium phosphate, tripotassium phosphate, and sodium methoxide. These alkali substances may be used alone or in combination of two or more thereof.

The acidic substance is not particularly limited, and examples thereof include adipic acid, benzoic acid, citric acid, glucono-δ-lactone, gluconic acid, DL-tartaric acid, L-tartaric acid, carbon dioxide, lactic acid, glacial acetic acid, disodium dihydrogen pyrophosphate, fumaric acid, monosodium fumarate, DL-malic acid, phosphoric acid, potassium dihydrogen phosphate, sodium dihydrogen phosphate, L-ascorbic acid, hydrochloric acid, L-glutamic acid, cinnamic acid, succinic acid, oxalic acid, and ammonium sulfate. These acidic substances may be used alone or in combination of two or more thereof.

In the step of distilling the extract liquid to remove the water and the organic solvent, water and/or an organic solvent may be added to promote removal of a specific organic solvent.

A treatment of bringing a purified product of a citrus oil obtained by the step of distilling the extract liquid to remove the water and the organic solvent into contact with a gas to further remove a specific organic solvent may be performed as necessary. At this time, the purified product of a citrus oil may be brought into contact with the gas as it is, or the purified product of a citrus oil may be added with water and/or an organic solvent and then brought into contact with the gas to promote removal of a specific organic solvent.

A method of bringing a purified product of a citrus oil into contact with a gas is not particularly limited, and a method of mixing a purified product of a citrus oil with a gas, a method of supplying a gas as bubbles to a purified product of a citrus oil, and the like are preferable. These methods may be used alone or in combination of two or more.

A treatment temperature at the time of bringing the purified product of a citrus oil into contact with a gas is not particularly limited, and is preferably 130° C. or lower.

The organic solvent to be added for promoting removal of a specific organic solvent is not particularly limited, and is preferably methanol, ethanol, 1-propanol, 2-propanol, 1-butanol, 2-butanol, propylene glycol, glycerin, acetone, methyl acetate, ethyl acetate, hexane, heptane, edible oils and fats, or middle chain fatty acid triacylglycerol (MCT). These organic solvents may be used alone or in combination of two or more thereof.

The gas to be brought into contact with the purified product of a citrus oil is not particularly limited, and is preferably air, hydrogen, oxygen, nitrogen, nitrous oxide, carbon dioxide, argon, helium, or water vapor. These gases may be used alone or in combination of two or more thereof.

### Fragrance or Flavor Composition

The fragrance or flavor composition according to the present invention contains the purified product of a citrus oil according to the present invention. A content of the purified product of a citrus oil according to the present invention in the fragrance or flavor composition according to the present invention can be appropriately determined depending on properties of the fragrance or flavor composition.

The fragrance or flavor composition according to the present invention may contain known fragrance or flavor components in addition to the purified product of a citrus oil according to the present invention.

The known fragrance or flavor component is not particularly limited, and examples thereof include terpene hydrocarbons such as α-pinene, β-pinene, limonene, and p-cymene, aliphatic alcohols such as octanol and p-tert-butylcyclohexanol. terpene alcohols such as menthol, citronellol, and geraniol, aromatic alcohols such as benzyl alcohol and phenylethyl alcohol, aliphatic aldehydes, terpene aldehydes, aromatic aldehydes, acetals, chain ketones, cyclic ketones such as damascone, β-ionone (ionone). and methylionone, terpene ketones such as carvone, menthone, isomenthone, and camphor, aromatic ketones such as acetophenone and raspberry ketone, ethers such as dibenzyl ether, oxides such as linalool oxide and rose oxide, musks such as cyclopentadecanolide and cyclohexadecanolide, lactones such as γ-nonalactone, γ-undecalactone, and coumarin, aliphatic esters such as acetate ester and propionate ester. and aromatic esters such as benzoate ester and phenylacetate ester. These fragrance or flavor components may be used alone or in combination of two or more thereof.

The fragrance or flavor composition according to the present invention may further contain a solvent or a fixative. The solvent and the fixative are not particularly limited, and examples thereof include rosin ester derivatives such as ethanol, isopropyl alcohol, ethylene glycol, propylene glycol, dipropylene glycol, butylene glycol, pentylene glycol, hexylene glycol, polyethylene glycol, diethyl phthalate, isopropyl myristate, triethyl cirtrate, benzyl benzoate, glycerin, triacetin, benzyl alcohol, paraffin, isoparaffin, and hercolyn, glycol ethers such as 3-methoxy-3-methyl-1-butanol. ethyl carbitol (diethylene glycol monoethyl ether), ethylene glycol monomethyl ether, propylene glycol monomethyl ether, ethylene glycol monoethyl ether, dipropylene glycol monomethyl ether, tripropylene glycol methyl ether, dipropylene glycol dimethyl ether, dipropylene glycol propyl ether, dipropylene glycol methyl ether acetate. and dipropylene glycol butyl ether. terpene resins such as pinene polymer. silicones such as cyclic silicone, edible fats and oils. triacylglycerol (MCT), and water. These solvents and fixatives may be used alone or in combination of two or more thereof.

The fragrance or flavor composition according to the present invention may further contain known components such as a higher alcohol, a surfactant, an antioxidant, an ultraviolet absorber, a chelating agent, a solubilizing agent, a stabilizing agent, a cooling agent, a preservative, an antibacterial agent, a fungicide, an antifungal agent, an insecticidal component, a dye, and a pH adjusting agent, as necessary. These known components may be used alone or in combination of two or more thereof.

The fragrance or flavor composition according to the present invention can be obtained, for example, by mixing and stirring the components, and may be subjected to heating or the like as desired. A content of each component can be appropriately adjusted.

### Beverage or Food Product, Fragrance or Cosmetic Product, Pharmaceutical Product or Oral Care Product

A beverage or food product, a fragrance or cosmetic product, a pharmaceutical product, or an oral care product according to the present invention contains the fragrance or flavor composition according to the present invention. The fragrance or flavor composition according to the present invention used in the beverage or food product, the fragrance or cosmetic product, the pharmaceutical product, or the oral care product according to the present invention can be appropriately determined depending on properties of the beverage or food product. the fragrance or cosmetic product, the pharmaceutical product or the oral care product.

A form of the beverage or food product, the fragrance or cosmetic product, the pharmaceutical product or the oral care product according to the present invention is not limited, and may be a liquid, a solid, a semi-solid, or a fluid.

The beverage or food product is not particularly limited, and examples thereof include liquid products such as fruit juice beverage, fruit beverage, vegetable beverage, carbonated beverage, sports drink, coffee beverage, tea, black tea, yogurt beverage, milk beverage, lactic acid bacteria beverage, nutritional drink, alcoholic beverage, non-alcoholic beverage, soup, and mentsuyu, candy, gum, tablet, gummy, jelly, chocolate, ice cream, sherbet, ham, sausage, snack, baked confectionery such as cookie and cake, cotton candy, bread, seasonings such as powder sauce, oils and fats such as butter and margarine, solid products such as edible sheet food, and fluid products such as curry, stew; hashed beef rice, sauce, dressing, raw cream, cream, jam, and liquid food.

The fragrance or cosmetic product is not particularly limited, and examples thereof include fragrance products (perfume, eau de parfum, eau de toilette, eau de cologne, and the like), foundation cosmetics (face-cleansing cream, banishing cream, cleansing cream, cold cream, massage cream, milky lotion, skin lotion, cosmetic lotion, pack, makeup removal, and the like), finish cosmetics (foundation, face powder, solid face powder, tantalum cam powder. lipstick, lip cream, bluish, eye liner, mascara, eye shadow; eyebrow pencil, eye pack, nail enamel, enamel remover, and the like), hair cosmetics (pomade, brilliantine, set lotion, hair stick, hair solid, hair oil, hair treatment, hair cream, hair tonic, hair liquid, hair spray, bandrine, revitalizing hair tonic, hair dye, and the like), sunscreen cosmetics (tanning products, sunscreen products, and the like), and pharmaceutical cosmetics (antiperspirant, after-shaving lotion, after-shaving gel, permanent wave agent, pharmaceutical soap, pharmaceutical shampoo, pharmaceutical skin cosmetics, and the like).

The pharmaceutical product is not particularly limited, and examples thereof include an oral medicine, and an external preparation (poultice or ointment).

The oral care product is not particularly limited, and examples thereof include tooth brushing powder, toothpaste, liquid toothpaste, mouthwash, gingival massage cream, local coating agent, troche, chewing gum, and mouth spray.

### EXAMPLES

Hereinafter, the present invention will be specifically described with reference to Examples, but the present invention is not limited to thereto.

### (Comparative Example 1) Lemon Cold Pressed Oil Concentrate

A commercially available lemon cold pressed oil (3,000 g) (citral concentration of 2.4 mass % as a main aroma component, linoleic acid concentration of 0.07 mass %, and sesquiterpene hydrocarbons concentration of 1.3 mass %) was subjected to a rectification treatment using a distillation column (filler: Sulzer packing (Sulzer chemtech)) under conditions of a treatment pressure of 0.6 kPa to 2.0 kPa and a treatment temperature of 50° C. to 70° C. to remove the terpene hydrocarbons as a distillate, thereby obtaining a distillation concentrate (400 g) in which oxygenated compounds important for a lemon aroma were concentrated.

Subsequently, 400 g of the distillation concentrate and 5000 g of a 85 mass % aqueous methanol solution of an extraction solvent were mixed, followed by stirring at a treatment temperature of 20° C. to 30° C. and then allowing to stand still to separate and remove the terpene hydrocarbons as an oil layer, thereby obtaining 5230 g of an extract liquid from which the oxygenated compounds were extracted.

Subsequently, 5230 g of the extract liquid from which the oxygenated compounds were extracted was subjected to a rectification treatment using a distillation column (filler: Helipack (TO-TOKU ENGINERING CO., LTD)) under conditions of a treatment pressure of 40 kPa to 70 kPa and a treatment temperature of 50° C. to 70° C. to remove the extraction solvent as a distillate. Thereafter, the obtained extract liquid concentrate was allowed to stand still to separate and remove a water layer, and the obtained oil layer was filtered to obtain 135 g of a lemon cold pressed oil concentrate (citral concentration of 44.4 mass % as a main aroma component, linoleic acid concentration of 1.38 mass %, and sesquiterpene hydrocarbons concentration of 2.3 mass %).

The purified product of a citrus oil did not satisfy a requirement “a content of linoleic acid with respect to 1 part by mass of citral as a main aroma component is 0.015 parts by mass or less”.

### (Example 1) Lemon Cold Pressed Oil Concentrate Obtained by Production Method Including Step of Performing Aqueous Alkali Solution Washing Treatment

The lemon cold pressed oil concentrate (100 g) according to Comparative Example 1 and a 5 mass % aqueous potassium carbonate solution (200 g) were mixed, followed by stirring at a treatment temperature of 20° C. to 30° C. and then allowing to stand still to separate and remove a water layer, thereby obtaining a product after the aqueous alkali solution washing treatment (93.2 g).

Finally, 93.2 g of the product after the aqueous alkali solution washing treatment and 200 g of a 1 mass % aqueous anhydrous citric acid solution were mixed and then allowed to stand still to separate and remove the water layer, and the obtained oil layer was filtered to obtain 84.1 g of a lemon cold pressed oil concentrate (citral concentration of 47.6 mass % as a main aroma component, linoleic acid concentration of 0.10 mass %, and sesquiterpene hydrocarbons concentration of 2.4 mass %).

The purified product of a citrus oil satisfied requirements “a content of linoleic acid with respect to 1 part by mass of citral as a main aroma component is 0.015 parts by mass or less” and “a content of sesquiterpene hydrocarbons is 20 mass % or less”.

### (Example 2) Lemon Cold Pressed Oil Concentrate Obtained by Production Method Including Step of Performing Aqueous Alkali Solution Washing Treatment

The lemon cold pressed oil concentrate (100 g) according to Comparative Example 1 and a 0.6 mass % aqueous potassium carbonate solution (200 g) were mixed, followed by stirring at a treatment temperature of 20° C. to 30° C. and then allowing to stand still to separate and remove a water layer, thereby obtaining a product after the aqueous alkali solution washing treatment (99 g).

Finally, 99 g of the product after the aqueous alkali solution washing treatment and 200 g of a 0.1 mass % aqueous anhydrous citric acid solution were mixed and then allowed to stand still to separate and remove the water layer, and the obtained oil layer was filtered to obtain 88.7 g of a lemon cold pressed oil concentrate (citral concentration of 46.3 mass % as a main aroma component, linoleic acid concentration of 0.68 mass %, and sesquiterpene hydrocarbons concentration of 2.4 mass %).

The purified product of a citrus oil satisfied requirements “a content of linoleic acid with respect to 1 part by mass of citral as a main aroma component is 0.015 parts by mass or less” and “a content of sesquiterpene hydrocarbons is 20 mass % or less”.

### (Comparative Example 2) Lemon Cold Pressed Oil Concentrate Obtained by Production Method Including Step of Performing Aqueous Alkali Solution Washing Treatment

The lemon cold pressed oil concentrate (100 g) according to Comparative Example 1 and a 0.25 mass % aqueous potassium carbonate solution (200 g) were mixed, followed by stirring at a treatment temperature of 20° C. to 30° C. and then allowing to stand still to separate and remove a water layer, thereby obtaining a product after the aqueous alkali solution washing treatment (99.5 g).

Finally, 99.5 g of the product after the aqueous alkali solution washing treatment and 200 g of a 0.1 mass % aqueous anhydrous citric acid solution were mixed and then allowed to stand still to separate and remove the water layer, and the obtained oil layer was filtered to obtain 89.5 g of a lemon cold pressed oil concentrate (citral concentration of 45.3 mass % as a main aroma component, linoleic acid concentration of 1.1 mass %, and sesquiterpene hydrocarbons concentration of 2.3 mass %).

The purified product of a citrus oil did not satisfy a requirement “a content of linoleic acid with respect to 1 part by mass of citral as a main aroma component is 0.015 parts by mass or less”.

### (Example 3) Lemon Cold Pressed Oil Concentrate Obtained by Production Method Including Step of Performing Ion Exchange Resin Treatment

An anion exchange resin (DIAION PA316L (Mitsubishi Chemical Corporation)) (25 g) subjected to a regeneration treatment with an aqueous sodium hydroxide solution and then substituted with water and a cation exchange resin (DIAION PK228L (Mitsubishi Chemical Corporation)) (10 g) subjected to a regeneration treatment with an aqueous anhydrous citric acid solution and then substituted with water were mixed and filled in a column, followed by removing water from a lower portion of the column.

Subsequently, an operation of passing 100 g (0.11 L) of the lemon cold pressed oil concentrate according to Comparative Example 1 through the column at a treatment temperature of 20° C. to 30° C. and a passing speed of 2000 g/hr was repeated five times to obtain 96.2 g of a product after the ion exchange resin treatment.

Finally, sodium sulfate was added to 96.2 g of the product after the ion exchange resin treatment to remove water, followed by filtration to obtain 94 g of a lemon cold pressed oil concentrate (citral concentration of 46.0 mass % as a main aroma component, linoleic acid concentration of 0.68 mass %, and sesquiterpene hydrocarbons concentration of 2.4 mass %).

The purified product of a citrus oil satisfied requirements “a content of linoleic acid with respect to 1 part by mass of citral as a main aroma component is 0.015 parts by mass or less” and “a content of sesquiterpene hydrocarbons is 20 mass % or less”.

### (Example 4) Lemon Cold Pressed Oil Concentrate Obtained by Production Method Including Step of Performing Ion Exchange Resin Treatment

An anion exchange resin (DIAION PA316L (Mitsubishi Chemical Corporation)) (25 g) subjected to a regeneration treatment with an aqueous sodium hydroxide solution and then substituted with water was filled in a column, followed by removing water from a lower portion of the column.

Subsequently, an operation of passing 100 g (0.11 L) of the lemon cold pressed oil concentrate according to Comparative Example 1 through the column at a treatment temperature of 20° C. to 30° C. and a passing speed of 2000 g/hr was repeated five times to obtain 97.0 g of a product after the ion exchange resin treatment.

Finally, sodium sulfate was added to 97.0 g of the product after the ion exchange resin treatment to remove water, followed by filtration to obtain 95.0 g of a lemon cold pressed oil concentrate (citral concentration of 46.1 mass % as a main aroma component, linoleic acid concentration of 0.67 mass %, and sesquiterpene hydrocarbons concentration of 2.4 mass %).

The purified product of a citrus oil satisfied requirements “a content of linoleic acid with respect to 1 part by mass of citral as a main aroma component is 0.015 parts by mass or less” and “a content of sesquiterpene hydrocarbons is 20 mass % or less”.

### (Example 5) Lemon Cold Pressed Oil Concentrate Obtained by Production Method Including Step of Performing Treatment in which Thin-Film Distillation and Liquid-Liquid Extraction are Combined

A commercially available lemon cold pressed oil (3000 g) (citral concentration of 2.4 mass % as a main aroma component, linoleic acid concentration of 0.07 mass %, and sesquiterpene hydrocarbons concentration of 1.3 mass %) was subjected to a rectification treatment using a distillation column (filler: Sulzer packing (Sulzer chemtech)) under conditions of a treatment pressure of 0.6 kPa to 2.0 kPa and a treatment temperature of 50° C. to 70° C. to remove the terpene hydrocarbons as a distillate, thereby obtaining a distillation concentrate (400 g) in which oxygenated compounds important for a lemon aroma were concentrated.

Subsequently, 400 g of the distillation concentrate was treated using a molecular distillation apparatus under conditions of a treatment pressure of 0.2 kPa to 1.0 kPa and a treatment temperature of 50° C. to 70° C. to recover a distillate (1) and a residue (1). Next, the residue (1) was treated under conditions of a treatment pressure of 0.1 kPa to 0.5 kPa and a treatment temperature of 50° C. to 70° C. to recover a distillate (2) and a residue (2). Next, the residue (2) was treated under conditions of a treatment pressure of 0.01 kPa to 0.09 kPa and a treatment temperature of 70° C. to 100° C. to recover a distillate (3) and a residue (3), and the distillates (1) to (3) were mixed to obtain 320 g of a thin-film distilled concentrate.

Subsequently, 320 g of the thin-film distilled concentrate and 4000 g of a 85 mass % aqueous methanol solution of an extraction solvent were mixed, followed by stirring at a treatment temperature of 20° C. to 30° C. and then allowing to stand still to separate and remove the terpene hydrocarbons as an oil layer, thereby obtaining 4180 g of an extract liquid from which the oxygenated compounds were extracted.

Subsequently, 4180 g of the extract liquid from which the oxygenated compounds were extracted was subjected to a rectification treatment using a distillation column (filler: Helipack (TO-TOKU ENGINERING CO., LTD)) under conditions of a treatment pressure of 40 kPa to 70 kPa and a treatment temperature of 50° C. to 70° C. to remove the extraction solvent as a distillate. Thereafter, the obtained extract liquid concentrate was allowed to stand still to separate and remove a water layer, and the obtained oil layer was filtered to obtain 100 g of a lemon cold pressed oil concentrate (citral concentration of 57.7 mass % as a main aroma component, linoleic acid concentration of 0) mass %, and sesquiterpene hydrocarbons concentration of 2.2 mass %).

The purified product of a citrus oil satisfied requirements “a content of linoleic acid with respect to 1 part by mass of citral as a main aroma component is 0.015 parts by mass or less” and “a content of sesquiterpene hydrocarbons is 20 mass % or less”.

### (Example 6) Lemon Cold Pressed Oil Concentrate Obtained by Production Method Including Step of Performing Treatment in which Thin-Film Distillation and Liquid-Liquid Extraction are Combined

The thin-film distilled concentrate (320 g) according to Example 5 and a 60 mass % aqueous methanol solution (6000 g) of an extraction solvent were mixed, followed by stirring at a treatment temperature of 20° C. to 30° C. and then allowing to stand still to separate and remove terpene hydrocarbons as an oil layer, thereby obtaining an extract liquid (6175 g) from which oxygenated compounds were extracted.

Subsequently, 6175 g of the extract liquid from which oxygenated compounds were extracted was subjected to a rectification treatment using a distillation column (filler: Helipack (TO-TOKU ENGINERING CO., LTD)) under conditions of a treatment pressure of 40 kPa to 70 kPa and a treatment temperature of 50° C. to 70° C. to remove the extraction solvent as a distillate. Thereafter, the obtained extract liquid concentrate was allowed to stand still to separate and remove a water layer, and the obtained oil layer was filtered to obtain 98 g of a lemon cold pressed oil concentrate (citral concentration of 59.5 mass % as a main aroma component, linoleic acid concentration of 0 mass %, and sesquiterpene hydrocarbons concentration of 2.0 mass %).

The purified product of a citrus oil satisfied requirements “a content of linoleic acid with respect to 1 part by mass of citral as a main aroma component is 0.015 parts by mass or less” and “a content of sesquiterpene hydrocarbons is 20 mass % or less”.

### (Example 7) Lemon Cold Pressed Oil Concentrate Obtained by Production Method Including Step of Performing Treatment in which Thin-Film Distillation and Liquid-Liquid Extraction are Combined

A commercially available lemon cold pressed oil (3000 g) (citral concentration of 2.4 mass % as a main aroma component, linoleic acid concentration of 0.07 mass %, and sesquiterpene hydrocarbons concentration of 1.3 mass %) was subjected to a rectification treatment using a distillation column (filler: Sulzer packing (Sulzer chemtech)) under conditions of a treatment pressure of 0.6 kPa to 2.0 kPa and a treatment temperature of 50° C. to 70° C. to remove the terpene hydrocarbons as a distillate, thereby obtaining a distillation concentrate (600 g) in which oxygenated compounds important for a lemon aroma were concentrated.

Subsequently, 600 g of the distillation concentrate was treated using a molecular distillation apparatus under conditions of a treatment pressure of 0.2 kPa to 1.0 kPa and a treatment temperature of 50° C. to 70° C. to recover a distillate (1) and a residue (1). Next, the residue (1) was treated under conditions of a treatment pressure of 0.1 kPa to 0.5 kPa and a treatment temperature of 50° C. to 70° C. to recover a distillate (2) and a residue (2). Next, the residue (2) was treated under conditions of a treatment pressure of 0.01 kPa to 0.09 kPa and a 30) treatment temperature of 70° C. to 100° C. to recover a distillate (3) and a residue (3), and the distillates (1) to (3) were mixed to obtain 510 g of a thin-film distilled concentrate.

Subsequently, 510 g of the thin-film distilled concentrate and 7650 g of a 70 mass % aqueous methanol solution of an extraction solvent were mixed, followed by stirring at a treatment temperature of 20° C. to 30° C. and then allowing to stand still to separate and remove the terpene hydrocarbons as an oil layer, thereby obtaining 7827 g of an extract liquid from which the oxygenated compounds were extracted.

Subsequently, 7827 g of the extract liquid from which the oxygenated compounds were extracted was subjected to a rectification treatment using a distillation column (filler: Helipack (TO-TOKU ENGINERING CO., LTD)) under conditions of a treatment pressure of 40 kPa to 70 kPa and a treatment temperature of 50° C. to 70° C. to remove the extraction solvent as a distillate. Thereafter, the obtained extract liquid concentrate was allowed to stand still to separate and remove a water layer, and the obtained oil layer was filtered to obtain 97 g of a lemon cold pressed oil concentrate (citral concentration of 58.3 mass % as a main aroma component, linoleic acid concentration of 0 mass %, and sesquiterpene hydrocarbons concentration of 2.1 mass %).

The purified product of a citrus oil satisfied requirements “a content of linoleic acid with respect to 1 part by mass of citral as a main aroma component is 0.015 parts by mass or less” and “a content of sesquiterpene hydrocarbons is 20 mass % or less”.

### (Example 8) Lemon Cold Pressed Oil Concentrate Obtained by Production Method Including Step of Performing Treatment in which Thin-Film Distillation and Liquid-Liquid Extraction are Combined

The thin-film distilled concentrate (320) g) according to Example 5 and a 80 mass % aqueous ethanol solution (4200 g) of an extraction solvent were mixed, followed by stirring at a treatment temperature of 20° C. to 30° C. and then allowing to stand still to separate and remove terpene hydrocarbons as an oil layer, thereby obtaining an extract liquid (4410 g) from which an oxygenated compounds were extracted.

Subsequently, 4410 g of the extract liquid from which the oxygenated compounds were extracted was subjected to a rectification treatment using a distillation column (filler: Helipack (TO-TOKU ENGINERING CO., LTD)) under conditions of a treatment pressure of 30 kPa to 60 kPa and a treatment temperature of 50° C. to 70° C. to remove the extraction solvent as a distillate. Thereafter, the obtained extract liquid concentrate was allowed to stand still to separate and remove a water layer, and the obtained oil layer was filtered to obtain 110 g of a lemon cold pressed oil concentrate (citral concentration of 54.4 mass % as a main aroma component, linoleic acid concentration of 0 mass %, and sesquiterpene hydrocarbons concentration of 5.1 mass %).

The purified product of a citrus oil satisfied requirements “a content of linoleic acid with respect to 1 part by mass of citral as a main aroma component is 0.015 parts by mass or less” and “a content of sesquiterpene hydrocarbons is 20 mass % or less”.

### (Example 9) Lemon Cold Pressed Oil Concentrate Obtained by Production Method Including Step of Performing Treatment in which Thin-Film Distillation and Liquid-Liquid Extraction are Combined

The thin-film distilled concentrate (320 g) according to Example 5 and a 60 mass % aqueous ethanol solution (6000 g) of an extraction solvent were mixed, followed by stirring at a treatment temperature of 20° C. to 30° C. and then allowing to stand still to separate and remove terpene hydrocarbons as an oil layer, thereby obtaining an extract liquid (6204 g) from which oxygenated compounds were extracted.

Subsequently, 6204 g of the extract liquid from which the oxygenated compounds were extracted was subjected to a rectification treatment using a distillation column (filler: Helipack (TO-TOKU ENGINERING CO., LTD)) under conditions of a treatment pressure of 30 kPa to 60 kPa and a treatment temperature of 50° C. to 70° C. to remove the extraction solvent as a distillate. Thereafter, the obtained extract liquid concentrate was allowed to stand still to separate and remove a water layer, and the obtained oil layer was filtered to obtain 107 g of a lemon cold pressed oil concentrate (citral concentration of 56.3 mass % as a main aroma component, linoleic acid concentration of 0) mass %, and sesquiterpene hydrocarbons concentration of 4.0 mass %).

The purified product of a citrus oil satisfied requirements “a content of linoleic acid with respect to 1 part by mass of citral as a main aroma component is 0.015 parts by mass or less” and “a content of sesquiterpene hydrocarbons is 20 mass % or less”.

### (Example 10) Lemon Cold Pressed Oil Concentrate Obtained by Production Method Including Step of Performing Treatment in which Thin-Film Distillation and Liquid-Liquid Extraction are Combined

The thin-film distilled concentrate (510) g) according to Example 7 and a 70 mass % aqueous ethanol solution (7650) g) of an extraction solvent were mixed, followed by stirring at a treatment temperature of 20° C. to 30° C. and then allowing to stand still to separate and remove terpene hydrocarbons as an oil layer, thereby obtaining an extract liquid (7857 g) from which oxygenated compounds were extracted.

Subsequently, 7857 g of the extract liquid from which the oxygenated compounds were extracted was subjected to a rectification treatment using a distillation column (filler: Helipack (TO-TOKU ENGINERING CO., LTD)) under conditions of a treatment pressure of 30 kPa to 60 kPa and a treatment temperature of 50° C. to 70° C. to remove the extraction solvent as a distillate. Thereafter, the obtained extract liquid concentrate was allowed to stand still to separate and remove a water layer, and the obtained oil layer was filtered to obtain 108 g of a lemon cold pressed oil concentrate (citral concentration of 55.0 mass % as a main aroma component, linoleic acid concentration of 0) mass %, and sesquiterpene hydrocarbons concentration of 4.5 mass %).

The purified product of a citrus oil satisfied requirements “a content of linoleic acid with respect to 1 part by mass of citral as a main aroma component is 0.015 parts by mass or less” and “a content of sesquiterpene hydrocarbons is 20 mass % or less”.

### (Comparative Example 3) Lemon Cold Pressed Oil Concentrate Obtained by Production Method Including Step of Performing Treatment in which Thin-Film Distillation and Aqueous Alkali Solution Washing Treatment are Combined

A commercially available lemon cold pressed oil (3000 g) (citral concentration of 2.4 mass % as a main aroma component, linoleic acid concentration of 0.07 mass %, and sesquiterpene concentration of 1.3 mass %) was subjected to a rectification treatment using a distillation column (filler: Sulzer packing (Sulzer chemtech)) under conditions of a treatment pressure of 0.3 kPa to 2.0 kPa and a treatment temperature of 50° C. to 100° C. to remove the terpene hydrocarbons as a distillate, thereby obtaining a distillation concentrate (260 g) in which oxygenated compounds important for a lemon aroma were concentrated.

Subsequently, 260 g of the distillation concentrate was treated using a molecular distillation apparatus under conditions of a treatment pressure of 0.1 kPa to 0.5 kPa and a treatment temperature of 50° C. to 70° C. to recover a distillate (1) and a residue (1). Next, the residue (1) was treated under conditions of a treatment pressure of 0.01 kPa to 0.09 kPa and a treatment temperature of 70° C. to 100° C. to recover a distillate (2) and a residue (2). Next, the residue (2) was treated under conditions of a treatment pressure of 0.005 kPa to 0.02 kPa and a treatment temperature of 90° C. to 120° C. to recover a distillate (3) and a residue (3), and the distillates (1) to (3) were mixed to obtain 195 g of a thin-film distilled concentrate.

Subsequently, 100 g of the thin-film distilled concentrate and 200 g of a 5 mass % aqueous potassium carbonate solution were mixed, followed by stirring at a treatment temperature of 20° C. to 30° C. and then allowing to stand still to separate and remove a water layer, thereby obtaining 94.0 g of a product after the aqueous alkali solution washing treatment.

Finally, 94.0 g of the product after the aqueous alkali solution washing treatment and 200 g of a 1 mass % aqueous anhydrous citric acid solution were mixed and then allowed to stand still to separate and remove the water layer, and the obtained oil layer was filtered to obtain 86.0 g of a lemon cold pressed oil concentrate (citral concentration of 39.3 mass % as a main aroma component, linoleic acid concentration of 0.05 mass %, and sesquiterpene hydrocarbons concentration of 21.3 mass %).

The purified product of a citrus oil satisfied a requirement “a content of linoleic acid with respect to 1 part by mass of citral as a main aroma component is 0.015 parts by mass or less”, but did not satisfy a requirement “a content of sesquiterpene hydrocarbons is 20 mass % or less”.

### (Comparative Example 4) Commercially Available Lemon Cold Pressed Oil

A commercially available lemon cold pressed oil (citral concentration of 2.4 mass % as a main aroma component, linoleic acid concentration of 0.07 mass %, and sesquiterpene hydrocarbons concentration of 1.3 mass %) was used as it was.

### (Example 11) Lemon Cold Pressed Oil Obtained by Production Method Including Step of Performing Aqueous Alkali Solution Washing Treatment

The commercially available lemon cold pressed oil (100 g) according to Comparative Example 4 and a 5 mass % aqueous potassium carbonate solution (200 g) were mixed, followed by stirring at a treatment temperature of 20° C. to 30° C. and then allowing to stand still to separate and remove a water layer, thereby obtaining a product after the aqueous alkali solution washing treatment (99 g).

Finally, 99 g of the product after the aqueous alkali solution washing treatment and 200 g of a 1 mass % aqueous anhydrous citric acid solution were mixed and then allowed to stand still to separate and remove the water layer, and the obtained oil layer was filtered to obtain 93 g of a lemon cold pressed oil (citral concentration of 2.4 mass % as a main aroma component, linoleic acid concentration of 0.006 mass %, and sesquiterpene hydrocarbons concentration of 1.3 mass %).

The purified product of a citrus oil satisfied requirements “a content of linoleic acid with respect to 1 part by mass of citral as a main aroma component is 0.015 parts by mass or less” and “a content of sesquiterpene hydrocarbons is 20 mass % or less”.

### (Comparative Example 5) Lemon Cold Pressed Oil Concentrate (Five Times Concentrate)

A commercially available lemon cold pressed oil (3000 g) (citral concentration of 2.4 mass % as a main aroma component, linoleic acid concentration of 0.07 mass %, and sesquiterpene hydrocarbons concentration of 1.3 mass %) was subjected to a rectification treatment using a distillation column (filler: Sulzer packing (Sulzer chemtech)) under conditions of a treatment pressure of 0.6 kPa to 2.0 kPa and a treatment temperature of 50° C. to 70° C. to remove the terpene hydrocarbons as a distillate, thereby obtaining a lemon cold pressed oil concentrate (600 g) (citral concentration of 12.0 mass % as a main aroma component, linoleic acid concentration of 0.35 mass %, and sesquiterpene hydrocarbons concentration of 6.5 mass %) in which oxygenated compounds important for a lemon aroma were concentrated.

The purified product of a citrus oil did not satisfy a requirement “a content of linoleic acid with respect to 1 part by mass of citral as a main aroma component is 0.015 parts by mass or less”.

### (Example 12) Lemon Cold Pressed Oil Concentrate Obtained by Production Method Including Step of Performing Aqueous Alkali Solution Washing Treatment (Five Times Concentrate)

The lemon cold pressed oil concentrate (100 g) according to Comparative Example 5 and a 5 mass % aqueous potassium carbonate solution (200 g) were mixed, followed by stirring at a treatment temperature of 20° C. to 30° C. and then allowing to stand still to separate and remove a water layer, thereby obtaining a product after the aqueous alkali solution washing treatment (76 g).

Finally, 76 g of the product after the aqueous alkali solution washing treatment and 200 g of a 1 mass % aqueous anhydrous citric acid solution were mixed and then allowed to stand still to separate and remove the water layer, and the obtained oil layer was filtered to obtain 70 g of a lemon cold pressed oil concentrate (citral concentration of 12.1 mass % as a main aroma component, linoleic acid concentration of 0.06 mass %, and sesquiterpene hydrocarbons concentration of 6.5 mass %).

The purified product of a citrus oil satisfied requirements “a content of linoleic acid with respect to 1 part by mass of citral as a main aroma component is 0.015 parts by mass or less” and “a content of sesquiterpene hydrocarbons is 20 mass % or less”.

### (Comparative Example 6) Orange Cold Pressed Oil Concentrate (Five Times Concentrate)

A commercially available orange cold pressed oil (3000 g) (linalool concentration of 0.50 mass % as a main aroma component, linoleic acid concentration of 0.072 mass %, and sesquiterpene hydrocarbons concentration of 0.03 mass %) was subjected to a rectification treatment using a distillation column (filler: Sulzer packing (Sulzer chemtech)) under conditions of a treatment pressure of 0.6 kPa to 2.0 kPa and a treatment temperature of 50° C. to 70° C. to remove the terpene hydrocarbons as a distillate, thereby obtaining an orange cold pressed oil concentrate (600 g) (linalool concentration of 2.51 mass % as a main aroma component, linoleic acid concentration of 0.36 mass %, and sesquiterpene hydrocarbons concentration of 0.15 mass %) in which oxygenated compounds important for an orange aroma were concentrated.

The purified product of a citrus oil did not satisfy a requirement “a content of linoleic acid with respect to 1 part by mass of linalool as a main aroma component was 0.063 parts by mass or less”.

### (Example 13) Orange Cold Pressed Oil Concentrate Obtained by Production Method Including Step of Performing Aqueous Alkali Solution Washing Treatment (Five Times Concentrate)

The orange cold pressed oil concentrate (100 g) according to Comparative Example 6 and a 5 mass % aqueous potassium carbonate solution (200 g) were mixed, followed by stirring at a treatment temperature of 20° C. to 30° C. and then allowing to stand still to separate and remove a water layer, thereby obtaining a product after the aqueous alkali solution washing treatment (89 g).

Finally, 89 g of the product after the aqueous alkali solution washing treatment and 200 g of a 1 mass % aqueous anhydrous citric acid solution were mixed and then allowed to stand still to separate and remove the water layer, and the obtained oil layer was filtered to obtain 84 g of an orange cold pressed oil concentrate (linalool concentration of 2.52 mass % as a main aroma component, linoleic acid concentration of 0.02 mass %, and sesquiterpene hydrocarbons concentration of 0.16 mass %).

The purified product of a citrus oil satisfied requirements “a content of linoleic acid with respect to 1 part by mass of linalool as a main aroma component is 0.063 parts by mass or less” and “a content of sesquiterpene hydrocarbons is 10 mass % or less”.

### (Comparative Example 7) Grapefruit Cold Pressed Oil Concentrate (Five Times Concentrate)

A commercially available grapefruit cold pressed oil (3000 g) (decanal concentration of 0.45 mass % as a main aroma component, linoleic acid concentration of 0.074 mass %, and sesquiterpene hydrocarbons concentration of 0.50 mass %) was subjected to a rectification treatment using a distillation column (filler: Sulzer packing (Sulzer chemtech)) under conditions of a treatment pressure of 0.6 kPa to 2.0 kPa and a treatment temperature of 50° C. to 70° C. to remove the terpene hydrocarbons as a distillate, thereby obtaining a grapefruit cold pressed oil concentrate (600 g) (decanal concentration of 2.24 mass % as a main aroma component, linoleic acid concentration of 0.37 mass %, and sesquiterpene hydrocarbons concentration of 2.5 mass %) in which oxygenated compounds important for a grapefruit aroma were concentrated.

The purified product of a citrus oil did not satisfy a requirement “a content of linoleic acid with respect to 1 part by mass of decanal as a main aroma component is 0.083 parts by mass or less”.

### (Example 14) Grapefruit Cold Pressed Oil Concentrate Obtained by Production Method Including Step of Performing Aqueous Alkali Solution Washing Treatment (Five Times Concentrate)

The grapefruit cold pressed oil concentrate (100 g) according to Comparative Example 7 and a 5 mass % aqueous potassium carbonate solution (200 g) were mixed, followed by stirring at a treatment temperature of 20° C. to 30° C. and then allowing to stand still to separate and remove a water layer, thereby obtaining a product after the aqueous alkali solution washing treatment (88 g).

Finally, 88 g of the product after the aqueous alkali solution washing treatment and 200 g of a 1 mass % aqueous anhydrous citric acid solution were mixed and then allowed to stand still to separate and remove the water layer, and the obtained oil layer was filtered to obtain 86 g of a grapefruit cold pressed oil concentrate (decanal concentration of 2.26 mass % as a main aroma component, linoleic acid concentration of 0.07 mass %, and sesquiterpene hydrocarbons concentration of 2.5 mass %).

The purified product of a citrus oil satisfied requirements “a content of linoleic acid with respect to 1 part by mass of decanal as a main aroma component is 0.083 parts by mass or less” and “a content of sesquiterpene hydrocarbons is 10 mass % or less”.

### (Comparative Example 8) Commercially Available Lime Cold Pressed Oil

A commercially available lime cold pressed oil (citral concentration of 3.95 mass % as a main aroma component, linoleic acid concentration of 0.073 mass %, and sesquiterpene hydrocarbons concentration of 4.5 mass %) was used as it was.

### (Example 15) Lime Cold Pressed Oil Obtained by Production Method Including Step of Performing Aqueous Alkali Solution Washing Treatment

The commercially available lime cold pressed oil (100 g) according to Comparative Example 8 and a 5 mass % aqueous potassium carbonate solution (200 g) were mixed, followed by stirring at a treatment temperature of 20° C. to 30° C. and then allowing to stand still to separate and remove a water layer, thereby obtaining a product after the aqueous alkali solution washing treatment (87 g).

Finally, 87 g of the product after the aqueous alkali solution washing treatment and 200 g of a 1 mass % aqueous anhydrous citric acid solution were mixed and then allowed to stand still to separate and remove the water layer, and the obtained oil layer was filtered to obtain 81 g of a lime cold pressed oil concentrate (citral concentration of 4.10 mass % as a main aroma component, linoleic acid concentration of 0.01 mass %, and sesquiterpene hydrocarbons concentration of 4.5 mass %).

The purified product of a citrus oil satisfied requirements “a content of linoleic acid with respect to 1 part by mass of citral is 0.008 parts by mass or less” and “a content of sesquiterpene hydrocarbons is 20 mass % or less”.

Hereinafter, effects of a citrus oil according to the present invention imparted with light stability while maintaining a fresh and preferable fragrance or flavor note derived from a raw material will be described with reference to Test Examples. A product sample blended with a product according to the present invention was prepared, and the effects were confirmed by sensory evaluation after the sample was subjected to a light exposure test.

### (Test Example 1) Sensory Evaluation: Lemon Cold Pressed Oil Concentrate

Flavor compositions 1 to 13 containing 1 mass % of the lemon cold pressed oil concentrates according to Comparative Examples 1 to 3 and Examples 1 to 10, respectively, were prepared in accordance with the formulation shown in Table 1.

Subsequently, acidic beverages blended with these flavor compositions were prepared in accordance with the formulation shown in Table 2 and were filled in a PET bottle. Thereafter, a heating sterilization treatment was performed at 80° C. for 10 minutes to prepare PET-bottled acidic beverages 1 to 13 containing 0.0005 mass % of the lemon cold pressed oil concentrates according to Comparative Examples 1 to 3 and Examples 1 to 10, respectively.

Subsequently, a light exposure test was performed on the PET-bottled acidic beverages. In the light exposure test, the PET-bottled acidic beverages were placed in a thermostatic chamber irradiated with light of 5000 lux to 6000 lux and then stored at 5° C. for one week. On the other hand, the PET-bottled acidic beverage 1 (blended with Comparative Example 1) placed in a light-shielded thermostatic chamber and then stored at 5° C. for 1 week was separately used as a control sample which was not exposed to light.

Subsequently, sensory evaluation was performed on the PET-bottled acidic beverages after the light exposure test. Four items of off-flavor, freshness, acidity, and palatability of the light-exposed PET-bottled acidic beverages 1 to 13 containing the lemon cold pressed oil concentrates according to Comparative Examples 1 to 3 and Examples 1 to 10, respectively, were evaluated on the basis of the control sample which was not exposed to light. In the evaluation, a randomly selected number was set as a sample name other than the control sample, and then information other than that an evaluation target was an acidic beverage was provided without being disclosed to an evaluator, so that it was impossible to predict what was included in the evaluation target and how the evaluation target was prepared.

The evaluation was performed by ten specialized panelists in a form of containing a beverage in their mouth, and results were shown in Table 3. A score of the control sample was set to five points for all the items, the four items of off-flavor, freshness, acidity, and palatability were evaluated as one point to five points in accordance with the evaluation criteria in Table 4, and an average value of ten specialized panelists was rounded off to the first decimal place and used as an evaluation point. When the evaluation points of all of the four items were four or more points close to the control sample, it was determined that generation of an abnormal odor was suppressed while maintaining a fresh and preferable flavor note originally possessed by an oil derived from a citrus peel as a raw material.

TABLE 1

| | Flavor | Flavor | Flavor | | | | |
|---|---|---|---|---|---|---|---|
| | composition 1 | composition 2 | composition 3 | Flavor | Flavor | Flavor | Flavor |
| | * blended | * blended | * blended | composition 4 | composition 5 | composition 6 | composition 7 |
| | with | with | with | * blended | * blended | * blended | * blended |
| Flavor | comparative | comparative | comparative | with | with | with | with |
| composition | example 1 | example 2 | example 3 | example 1 | example 2 | example 3 | example 4 |
| (Component) | (Blending | (Blending | (Blending | (Blending | (Blending | (Blending | (Blending |
| | amount/g) | amount/g) | amount/g) | amount/g) | amount/g) | amount/g) | amount/g) |
| Comparative | 0.1 | — | — | — | — | — | — |
| example 1 | | | | | | | |
| Comparative | — | 0.1 | — | — | — | — | — |
| example 2 | | | | | | | |
| Comparative | — | — | 0.1 | — | — | — | — |
| example 3 | | | | | | | |
| Example 1 | — | — | — | 0.1 | — | — | — |
| Example 2 | — | — | — | — | 0.1 | — | — |
| Example 3 | — | — | — | — | | 0.1 | — |
| Example 4 | — | — | — | — | — | — | 0.1 |
| 95 vol % ethanol | 9.9 | 9.9 | 9.9 | 9.9 | 9.9 | 9.9 | 9.9 |
| Total | 10.0 | 10.0 | 10.0 | 10.0 | 10.0 | 10.0 | 10.0 |

| | Flavor | Flavor | Flavor | Flavor | Flavor | Flavor |
|---|---|---|---|---|---|---|
| | composition 8 | composition 9 | composition 10 | composition 11 | composition 12 | composition 13 |
| | * blended | * blended | * blended | * blended | * blended | * blended |
| Flavor | with | with | with | with | with | with |
| composition | example 5 | example 6 | example 7 | example 8 | example 9 | example 10 |
| (Component) | (Blending | (Blending | (Blending | (Blending | (Blending | (Blending |
| | amount/g) | amount/g) | amount/g) | amount/g) | amount/g) | amount/g) |
| Example 5 | 0.1 | — | — | — | — | — |
| Example 6 | — | 0.1 | — | — | — | — |
| Example 7 | — | — | 0.1 | — | — | — |
| Example 8 | — | — | — | 0.1 | — | — |
| Example 9 | — | — | — | — | 0.1 | — |
| Example 10 | — | — | — | — | — | 0.1 |
| 95 vol % ethanol | 9.9 | 9.9 | 9.9 | 9.9 | 9.9 | 9.9 |
| Total | 10.0 | 10.0 | 10.0 | 10.0 | 10.0 | 10.0 |

TABLE 2

| | Acidic | Acidic | Acidic | | | | |
|---|---|---|---|---|---|---|---|
| | beverage 1 | beverage 2 | beverage 3 | Acidic | Acidic | Acidic | Acidic |
| | * blended | * blended | * blended | beverage 4 | beverage 5 | beverage 6 | beverage 7 |
| | with | with | with | * blended | * blended | * blended | * blended |
| | comparative | comparative | comparative | with | with | with | with |
| Acidic beverage | example 1 | example 2 | example 3 | example 1 | example 2 | example 3 | example 4 |
| (Component) | (Blending | (Blending | (Blending | (Blending | (Blending | (Blending | (Blending |
| | amount/g) | amount/g) | amount/g) | amount/g) | amount/g) | amount/g) | amount/g) |
| Flavor composition 1 | 0.5 | — | — | — | — | — | — |
| Flavor composition 2 | — | 0.5 | — | — | — | — | — |
| Flavor composition 3 | — | — | 0.5 | — | — | — | — |
| Flavor composition 4 | — | — | — | 0.5 | — | — | — |
| Flavor composition 5 | — | — | — | — | 0.5 | — | — |
| Flavor composition 6 | — | — | — | — | — | 0.5 | — |
| Flavor composition 7 | — | — | — | — | — | — | 0.5 |
| Granulated sugar | 30.0 | 30.0 | 30.0 | 30.0 | 30.0 | 30.0 | 30.0 |
| Anhydrous citric acid | 3.0 | 3.0 | 3.0 | 3.0 | 3.0 | 3.0 | 3.0 |
| Ion exchange water | 966.5 | 966.5 | 966.5 | 966.5 | 966.5 | 966.5 | 966.5 |
| Total | 1000.0 | 1000.0 | 1000.0 | 1000.0 | 1000.0 | 1000.0 | 1000.0 |

| | Acidic | Acidic | Acidic | Acidic | Acidic | Acidic |
|---|---|---|---|---|---|---|
| | beverage 8 | beverage 9 | beverage 10 | beverage 11 | beverage 12 | beverage 13 |
| | * blended | * blended | * blended | * blended | * blended | * blended |
| | with | with | with | with | with | with |
| Acidic beverage | example 5 | example 6 | example 7 | example 8 | example 9 | example 10 |
| (Component) | (Blending | (Blending | (Blending | (Blending | (Blending | (Blending |
| | amount/g) | amount/g) | amount/g) | amount/g) | amount/g) | amount/g) |
| Flavor composition 8 | 0.5 | — | — | — | — | — |
| Flavor composition 9 | — | 0.5 | — | — | — | — |
| Flavor composition 10 | — | — | 0.5 | — | — | — |
| Flavor composition 11 | — | — | — | 0.5 | — | — |
| Flavor composition 12 | — | — | — | — | 0.5 | — |
| Flavor composition 13 | — | — | — | — | — | 0.5 |
| Granulated sugar | 30.0 | 30.0 | 30.0 | 30.0 | 30.0 | 30.0 |
| Anhydrous citric acid | 3.0 | 3.0 | 3.0 | 3.0 | 3.0 | 3.0 |
| Ion exchange water | 966.5 | 966.5 | 966.5 | 966.5 | 966.5 | 966.5 |
| Total | 1000.0 | 1000.0 | 1000.0 | 1000.0 | 1000.0 | 1000.0 |

TABLE 3

| | | | | | | Suppress generation of |
|---|---|---|---|---|---|---|
| | | | | | | abnormal odor and maintain |
| | Light | Score | | | | flavor note derived from raw |
| Sample | exposure | Off-flavor | Freshness | Acidity | Palatability | material |
| Control | No | 5.0 | 5.0 | 5.0 | 5.0 | |
| Acidic beverage 1 | Yes | 2.8 | 2.3 | 4.3 | 2.3 | x |
| (blended with comparative example 1) | | | | | | |
| Acidic beverage 2 | Yes | 3.5 | 3.5 | 4.3 | 3.0 | x |
| (blended with comparative example 2) | | | | | | |
| Acidic beverage 3 | Yes | 4.5 | 3.3 | 4.2 | 3.3 | x |
| (blended with comparative example 3) | | | | | | |
| Acidic beverage 4 | Yes | 5.0 | 4.8 | 4.3 | 5.0 | ∘ |
| (blended with example 1) | | | | | | |
| Acidic beverage 5 | Yes | 4.3 | 4.1 | 4.3 | 4.3 | ∘ |
| (blended with example 2) | | | | | | |
| Acidic beverage 6 | Yes | 4.2 | 4.1 | 4.2 | 4.2 | ∘ |
| (blended with example 3) | | | | | | |
| Acidic beverage 7 | Yes | 4.2 | 4.1 | 4.2 | 4.1 | ∘ |
| (blended with example 4) | | | | | | |
| Acidic beverage 8 | Yes | 4.9 | 4.8 | 4.3 | 4.9 | ∘ |
| (blended with example 5) | | | | | | |
| Acidic beverage 9 | Yes | 4.9 | 4.9 | 4.5 | 4.9 | ∘ |
| (blended with example 6) | | | | | | |
| Acidic beverage 10 | Yes | 5.0 | 4.8 | 4.4 | 4.8 | ∘ |
| (blended with example 7) | | | | | | |
| Acidic beverage 11 | Yes | 5.0 | 4.7 | 4.4 | 4.9 | ∘ |
| (blended with example 8) | | | | | | |
| Acidic beverage 12 | Yes | 4.9 | 4.8 | 4.3 | 4.8 | ∘ |
| (blended with example 9) | | | | | | |
| Acidic beverage 13 | Yes | 4.9 | 4.8 | 4.5 | 4.9 | ∘ |
| (blended with example 10) | | | | | | |

TABLE 4

| | | | | |
|---|---|---|---|---|
| Off-flavor | Freshness | Acidity | Palatability | Score |

| | | |
|---|---|---|
| Equivalent to control | Equivalent to control | Five points |
| Slightly strong | Slightly weak | Four points |
| Relatively strong | Relatively weak | Three points |
| Strong | Weak | Two points |
| Significantly strong | Significantly weak | One point |

As shown in Table 3, regarding the PET-bottled acidic beverages blended with the lemon cold pressed oil concentrates according to Examples 1 to 10, four or more scores were obtained for all items and generation of an abnormal odor was suppressed while maintaining a fresh and preferable flavor note originally possessed by an oil derived from a citrus peel as a raw material as compared with the PET-bottled acidic beverages blended with the lemon cold pressed oil concentrates according to Comparative Example 1 in which a linoleic acid reduction treatment was not performed, and therefore, a content ratio of linoleic acid with respect to a content of citral as a main aroma component was out of a specified range, Comparative Example 2 in which a linoleic acid reduction treatment was performed, but a content ratio of linoleic acid with respect to a content of citral as a main aroma component was out of a specified range, and Comparative Example 3 in which a linoleic acid reduction treatment was performed, and a content of linoleic acid with respect to a content of citral as a main aroma component was in a specified range, but a content ratio of sesquiterpene hydrocarbons was out of a specified range.

### (Test Example 2) Sensory Evaluation: Lemon Cold Pressed Oil

Flavor compositions 14 and 15 containing 10 mass % of the lemon cold pressed oils according to Comparative Example 4 and Example 11, respectively, were prepared in accordance with the formulation shown in Table 5.

Subsequently, acid beverage blended with these flavor compositions were prepared in accordance with the formulation shown in Table 6 and were filled in a PET bottle. Thereafter, a heating sterilization treatment was performed at 80° C. for 10 minutes to prepare PET-bottled acidic beverages 14 and 15 containing 0.01 mass % of the lemon cold pressed oils according to Comparative Example 4 and Example 11, respectively.

Subsequently, a light exposure test was performed on the PET-bottled acidic beverages in the same manner as in Test Example 1. On the other hand, the PET-bottled acidic beverage 14 (blended with Comparative Example 4) placed in a light-shielded thermostatic chamber and then stored at 5° C. for 1 week was separately used as a control sample which was not exposed to light.

Subsequently, sensory evaluation was performed on the PET-bottled acidic beverages after the light exposure test. Four items of off-flavor, freshness, acidity, and palatability of the light-exposed PET-bottled acidic beverages 14 and 15 containing the lemon cold pressed oils according to Comparative Example 4 and Example 11, respectively, were evaluated on the basis of the control sample which was not exposed to light. In the evaluation, a randomly selected number was set as a sample name other than the control sample, and then information other than that an evaluation target was an acidic beverage was provided without being disclosed to an evaluator, so that it was impossible to predict what was included in the evaluation target and how the evaluation target was prepared.

The evaluation was performed by ten specialized panelists in a form of containing a beverage in their mouth, and results were shown in Table 7. Evaluation criteria for determining that generation of an abnormal odor was suppressed while maintaining a fresh and preferable flavor note originally possessed by an oil derived from a citrus peel as a raw material were the same as those in Test Example 1.

TABLE 5

| | Flavor composition 14 | Flavor composition 15 |
|---|---|---|
| | * blended with | * blended with |
| Flavor composition | comparative example 4 | example 11 |
| (Component) | (Blending amount/g) | (Blending amount/g) |
| Comparative example 4 | 1.0 | — |
| Example 11 | — | 1.0 |
| 95 vol % ethanol | 9.0 | 9.0 |
| Total | 10.0 | 10.0 |

TABLE 6

| | Acidic beverage 14 | Acidic beverage 15 |
|---|---|---|
| | * blended with | * blended with |
| Acidic beverage | comparative example 4 | example 11 |
| (Component) | (Blending amount/g) | (Blending amount/g) |
| Flavor composition 14 | 1.0 | — |
| Flavor composition 15 | — | 1.0 |
| Granulated sugar | 30.0 | 30.0 |
| Anhydrous citric acid | 3.0 | 3.0 |
| Ion exchange water | 966.0 | 966.0 |
| Total | 1,000.0 | 1,000.0 |

TABLE 7

| | | | | | | Suppress generation of |
|---|---|---|---|---|---|---|
| | | | | | | abnormal odor and maintain |
| | Light | Score | | | | flavor note derived from raw |
| Sample | exposure | Off-flavor | Freshness | Acidity | Palatability | material |
| Control | No | 5.0 | 5.0 | 5.0 | 5.0 | |
| Acidic beverage 14 | Yes | 2.1 | 2.1 | 3.9 | 2.0 | x |
| (blended with comparative example 4) | | | | | | |
| Acidic beverage 15 | Yes | 4.2 | 4.1 | 4.1 | 4.2 | ∘ |
| (blended with example 11) | | | | | | |

As shown in Table 7, regarding the PET-bottled acidic beverage blended with the lemon cold pressed oil according to Example 11, four or more scores were obtained for all items and generation of an abnormal odor was suppressed while maintaining a fresh and preferable flavor note originally possessed by an oil derived from a citrus peel as a raw material as compared with the PET-bottled acidic beverage blended with Comparative Example 4 in which a linoleic acid reduction treatment was not performed, and therefore, a content ratio of linoleic acid with respect to a content of citral as a main aroma component was out of a specified range.

### (Test Example 3) Sensory Evaluation: Lemon Cold Pressed Oil (Five Times Concentrate)

Flavor compositions 16 and 17 containing 2 mass % of the lemon cold pressed oil concentrates according to Comparative Example 5 and Example 12, respectively, were prepared in accordance with the formulation shown in Table 8.

Subsequently, acidic beverages blended with these flavor compositions were prepared in accordance with the formulation shown in Table 9 and were filled in a PET bottle. Thereafter, a heating sterilization treatment was performed at 80° C. for 10 minutes to prepare PET-bottled acidic beverages 16 and 17 containing 0.002 mass % of the lemon cold pressed oil concentrates according to Comparative Example 5 and Example 12, respectively.

Subsequently, a light exposure test was performed on the PET-bottled acidic beverages in the same manner as in Test Example 1. On the other hand, the PET-bottled acidic beverage 16 (blended with Comparative Example 5) placed in a light-shielded thermostatic chamber and then stored at 5° C. for 1 week was separately used as a control sample which was not exposed to light.

Subsequently, sensory evaluation was performed on the PET-bottled acidic beverages after the light exposure test. Four items of off-flavor, freshness, acidity, and palatability of the light-exposed PET-bottled acidic beverages 16 and 17 containing the lemon cold pressed oil concentrates according to Comparative Example 5 and Example 12, respectively, were evaluated on the basis of the control sample which was not exposed to light. In the evaluation, a randomly selected number was set as a sample name other than the control sample, and then information other than that an evaluation target was an acidic beverage was provided without being disclosed to an evaluator, so that it was impossible to predict what was included in the evaluation target and how the evaluation target was prepared.

The evaluation was performed by ten specialized panelists in a form of containing a beverage in their mouth, and results were shown in Table 10. Evaluation criteria for determining that generation of an abnormal odor was suppressed while maintaining a fresh and preferable flavor note originally possessed by an oil derived from a citrus peel as a raw material were the same as those in Test Example 1.

TABLE 8

| | Flavor composition 16 | Flavor composition 17 |
|---|---|---|
| | * blended with | * blended with |
| Flavor composition | comparative example 5 | example 12 |
| (Component) | (Blending amount/g) | (Blending amount/g) |
| Comparative example 5 | 0.2 | — |
| Example 12 | — | 0.2 |
| 95 vol % ethanol | 9.8 | 9.8 |
| Total | 10.0 | 10.0 |

TABLE 9

| | Acidic beverage 16 | Acidic beverage 17 |
|---|---|---|
| | * blended with | * blended with |
| Acidic beverage | comparative example 5 | example 12 |
| (Component) | (Blending amount/g) | (Blending amount/g) |
| Flavor composition 16 | 1.0 | — |
| Flavor composition 17 | — | 1.0 |
| Granulated sugar | 30.0 | 30.0 |
| Anhydrous citric acid | 3.0 | 3.0 |
| Ion exchange water | 966.0 | 966.0 |
| Total | 1,000.0 | 1,000.0 |

TABLE 10

| | | | | | | Suppress generation of |
|---|---|---|---|---|---|---|
| | | | | | | abnormal odor and maintain |
| | Light | Score | | | | flavor note derived from raw |
| Sample | exposure | Off-flavor | Freshness | Acidity | Palatability | material |
| Control | No | 5.0 | 5.0 | 5.0 | 5.0 | |
| Acidic beverage 16 | Yes | 2.3 | 2.2 | 4.0 | 2.2 | x |
| (blended with comparative example 5) | | | | | | |
| Acidic beverage 17 | Yes | 4.3 | 4.3 | 4.1 | 4.3 | ∘ |
| (blended with example 12) | | | | | | |

As shown in Table 10, regarding the PET-bottled acidic beverage blended with the lemon cold pressed oil concentrate according to Example 12, four or more scores were obtained for all items and generation of an abnormal odor was suppressed while maintaining a fresh and preferable flavor note originally possessed by an oil derived from a citrus peel as a raw material as compared with the PET-bottled acidic beverage blended with Comparative Example 5 in which a linoleic acid reduction treatment was not performed, and therefore, a content ratio of linoleic acid with respect to a content of citral as a main aroma component was out of a specified range.

### (Test Example 4) Sensory Evaluation: Orange Cold Pressed Oil (Five Times Concentrate)

Flavor compositions 18 and 19 containing 2 mass % of the orange cold pressed oil concentrates according to Comparative Example 6 and Example 13, respectively, were prepared in accordance with the formulation shown in Table 11.

Subsequently, acidic beverages blended with these flavor compositions were prepared in accordance with the formulation shown in Table 12 and were filled in a PET bottle. Thereafter, a heating sterilization treatment was performed at 80° C. for 10 minutes to prepare PET-bottled acidic beverages 18 and 19 containing 0.002 mass % of the orange cold pressed oil concentrates according to Comparative Example 6 and Example 13, respectively.

Subsequently, a light exposure test was performed on the PET-bottled acidic beverages in the same manner as in Test Example 1. On the other hand, the PET-bottled acidic beverage 18 (blended with Comparative Example 6) placed in a light-shielded thermostatic chamber and then stored at 5° C. for 1 week was separately used as a control sample which was not exposed to light.

Subsequently, sensory evaluation was performed on the PET-bottled acidic beverages after the light exposure test. Four items of off-flavor, freshness, acidity, and palatability of the light-exposed PET-bottled acidic beverages 18 and 19 containing the orange cold pressed oil concentrates according to Comparative Example 6 and Example 13, respectively, were evaluated on the basis of the control sample which was not exposed to light. In the evaluation, a randomly selected number was set as a sample name other than the control sample, and then information other than that an evaluation target was an acidic beverage was provided without being disclosed to an evaluator, so that it was impossible to predict what was included in the evaluation target and how the evaluation target was prepared.

The evaluation was performed by ten specialized panelists in a form of containing a beverage in their mouth, and results were shown in Table 13. Evaluation criteria for determining that generation of an abnormal odor was suppressed while maintaining a fresh and preferable flavor note originally possessed by an oil derived from a citrus peel as a raw material were the same as those in Test Example 1.

TABLE 11

| | Flavor composition 18 | Flavor composition 19 |
|---|---|---|
| | * blended with | * blended with |
| Flavor composition | comparative example 6 | example 13 |
| (Component) | (Blending amount/g) | (Blending amount/g) |
| Comparative example 6 | 0.2 | — |
| Example 13 | — | 0.2 |
| 95 vol % ethanol | 9.8 | 9.8 |
| Total | 10.0 | 10.0 |

TABLE 12

| | Acidic beverage 18 | Acidic beverage 19 |
|---|---|---|
| | * blended with | * blended with |
| Acidic beverage | comparative example 6 | example 13 |
| (Component) | (Blending amount/g) | (Blending amount/g) |
| Flavor composition 18 | 1.0 | — |
| Flavor composition 19 | — | 1.0 |
| Granulated sugar | 30.0 | 30.0 |
| Anhydrous citric acid | 3.0 | 3.0 |
| Ion exchange water | 966.0 | 966.0 |
| Total | 1,000.0 | 1,000.0 |

TABLE 13

| | | | | | | Suppress generation of |
|---|---|---|---|---|---|---|
| | | | | | | abnormal odor and maintain |
| | Light | Score | | | | flavor note derived from raw |
| Sample | exposure | Off-flavor | Freshness | Acidity | Palatability | material |
| Control | No | 5.0 | 5.0 | 5.0 | 5.0 | |
| Acidic beverage 18 | Yes | 2.5 | 2.4 | 4.1 | 2.4 | x |
| (blended with comparative example 6) | | | | | | |
| Acidic beverage 19 | Yes | 4.2 | 4.3 | 4.2 | 4.3 | ∘ |
| (blended with example 13) | | | | | | |

As shown in Table 13, regarding the PET-bottled acidic beverage blended with the orange cold pressed oil concentrate according to Example 13, four or more scores were obtained for all items and generation of an abnormal odor was suppressed while maintaining a fresh and preferable flavor note originally possessed by an oil derived from a citrus peel as a raw material as compared with the PET-bottled acidic beverage blended with Comparative Example 6 in which as a linoleic acid reduction treatment was not performed, and therefore, a content ratio of linoleic acid with respect to a content of linalool as a main aroma component was out of a specified range.

### (Test Example 5) Sensory Evaluation: Grapefruit Cold Pressed Oil (Five Times Concentrate)

Flavor compositions 20 and 21 containing 2 mass % of the grapefruit cold pressed oil concentrates according to Comparative Example 7 and Example 14, respectively, were prepared in accordance with the formulation shown in Table 14.

Subsequently, acidic beverages blended with these flavor compositions were prepared in accordance with the formulation shown in Table 15 and were filled in a PET bottle. Thereafter, a heating sterilization treatment was performed at 80° C. for 10 minutes to prepare PET-bottled acidic beverages 20 and 21 containing 0.002 mass % of the grapefruit cold pressed oil concentrates according to Comparative Example 7 and Example 14, respectively.

Subsequently, a light exposure test was performed on the PET-bottled acidic beverages in the same manner as in Test Example 1. On the other hand, the PET-bottled acidic beverage 20 (blended with Comparative Example 7) placed in a light-shielded thermostatic chamber and then stored at 5° C. for 1 week was separately used as a control sample which was not exposed to light.

Subsequently, sensory evaluation was performed on the PET-bottled acidic beverages after the light exposure test. Four items of off-flavor, freshness, acidity, and palatability of the light-exposed PET-bottled acidic beverages 20 and 21 containing the grapefruit cold pressed oil concentrates according to Comparative Example 7 and Example 14, respectively, were evaluated on the basis of the control sample which was not exposed to light. In the evaluation, a randomly selected number was set as a sample name other than the control sample, and then information other than that an evaluation target was an acidic beverage was provided without being disclosed to an evaluator, so that it was impossible to predict what was included in the evaluation target and how the evaluation target was prepared.

The evaluation was performed by ten specialized panelists in a form of containing a beverage in their mouth, and results were shown in Table 16. Evaluation criteria for determining that generation of an abnormal odor was suppressed while maintaining a fresh and preferable flavor note originally possessed by an oil derived from a citrus peel as a raw material were the same as those in Test Example 1.

TABLE 14

| | Flavor composition 20 | Flavor composition 21 |
|---|---|---|
| | * blended with | * blended with |
| Flavor composition | comparative example 7 | example 14 |
| (Component) | (Blending amount/g) | (Blending amount/g) |
| Comparative example 7 | 0.2 | — |
| Example 14 | — | 0.2 |
| 95 vol % ethanol | 9.8 | 9.8 |
| Total | 10.0 | 10.0 |

TABLE 15

| | Acidic beverage 20 | Acidic beverage 21 |
|---|---|---|
| | * blended with | * blended with |
| Acidic beverage | comparative example 7 | example 14 |
| (Component) | (Blending amount/g) | (Blending amount/g) |
| Flavor composition 20 | 1.0 | — |
| Flavor composition 21 | — | 1.0 |
| Granulated sugar | 30.0 | 30.0 |
| Anhydrous citric acid | 3.0 | 3.0 |
| Ion exchange water | 966.0 | 966.0 |
| Total | 1,000.0 | 1,000.0 |

TABLE 16

| | | | | | | Suppress generation of |
|---|---|---|---|---|---|---|
| | | Score | | | | abnormal odor and maintain |
| | Light | Off- | | | | flavor note derived from raw |
| Sample | exposure | flavor | Freshness | Acidity | Palatability | material |
| Control | No | 5.0 | 5.0 | 5.0 | 5.0 | |
| Acidic beverage 20 | Yes | 2.5 | 2.5 | 4.0 | 2.3 | x |
| (blended with comparative example 7) | | | | | | |
| Acidic beverage 21 | Yes | 4.3 | 4.1 | 4.3 | 4.3 | ∘ |
| (blended with example 14) | | | | | | |

As shown in Table 16, regarding the PET-bottled acidic beverage blended with the grapefruit cold pressed oil concentrate according to Example 14, four or more scores were obtained for all items and generation of an abnormal odor was suppressed while maintaining a fresh and preferable flavor note originally possessed by an oil derived from a citrus peel as a raw material as compared with the PET-bottled acidic beverage blended with Comparative Example 7 in which a linoleic acid reduction treatment was not performed, and therefore, a content ratio of linoleic acid with respect to a content of decanal as a main aroma component was out of a specified range.

### (Test Example 6) Sensory Evaluation: Lime Cold Pressed Oil

Flavor compositions 22 and 23 containing 10 mass % of the lime cold pressed oils according to Comparative Example 8 and Example 15, respectively, were prepared in accordance with the formulation shown in Table 17.

Subsequently, acidic beverages blended with these flavor compositions were prepared in accordance with the formulation shown in Table 18 and were filled in a PET bottle. Thereafter, a heating sterilization treatment was performed at 80° C. for 10 minutes to prepare PET-bottled acidic beverages 22 and 23 containing 0.01 mass % of the lime cold pressed oils according to Comparative Example 8 and Example 15, respectively.

Subsequently, a light exposure test was performed on the PET-bottled acidic beverages in the same manner as in Test Example 1. On the other hand, the PET-bottled acidic beverage 22 (blended with Comparative Example 8) placed in a light-shielded thermostatic chamber and then stored at 5° C. for 1 week was separately used as a control sample which was not exposed to light.

Subsequently, sensory evaluation was performed on the PET-bottled acidic beverages after the light exposure test. Four items of off-flavor, freshness, acidity, and palatability of the light-exposed PET-bottled acidic beverages 22 and 23 containing the lime cold pressed oils according to Comparative Example 8 and Example 15, respectively, were evaluated on the basis of the control sample which was not exposed to light. In the evaluation, a randomly selected number was set as a sample name other than the control sample, and then information other than that an evaluation target was an acidic beverage was provided without being disclosed to an evaluator, so that it was impossible to predict what was included in the evaluation target and how the evaluation target was prepared.

The evaluation was performed by ten specialized panelists in a form of containing a beverage in their mouth, and results were shown in Table 19. Evaluation criteria for determining that generation of an abnormal odor was suppressed while maintaining a fresh and preferable flavor note originally possessed by an oil derived from a citrus peel as a raw material were the same as those in Test Example 1.

TABLE 17

| | Flavor composition 22 | Flavor composition 23 |
|---|---|---|
| | * blended with | * blended with |
| Flavor composition | comparative example 8 | example 15 |
| (Component) | (Blending amount/g) | (Blending amount/g) |
| Comparative example 8 | 1.0 | — |
| Example 15 | — | 1.0 |
| 95 vol % ethanol | 9.0 | 9.0 |
| Total | 10.0 | 10.0 |

TABLE 18

| | Acidic beverage 22 | Acidic beverage 23 |
|---|---|---|
| | * blended with | * blended with |
| Acidic beverage | comparative example 8 | example 15 |
| (Component) | (Blending amount/g) | (Blending amount/g) |
| Flavor composition 22 | 1.0 | — |
| Flavor composition 23 | — | 1.0 |
| Granulated sugar | 30.0 | 30.0 |
| Anhydrous citric acid | 3.0 | 3.0 |
| Ion exchange water | 966.0 | 966.0 |
| Total | 1,000.0 | 1,000.0 |

TABLE 19

| | | | | | | Suppress generation of |
|---|---|---|---|---|---|---|
| | | | | | | abnormal odor and maintain |
| | Light | Score | | | | flavor note derived from raw |
| Sample | exposure | Off-flavor | Freshness | Acidity | Palatability | material |
| Control | No | 5.0 | 5.0 | 5.0 | 5.0 | |
| Acidic beverage 22 | Yes | 2.3 | 2.1 | 4.0 | 2.1 | x |
| (blended with comparative example 8) | | | | | | |
| Acidic beverage 23 | Yes | 4.1 | 4.3 | 4.2 | 4.3 | ∘ |
| (blended with example 15) | | | | | | |

As shown in Table 19, regarding the PET-bottled acidic beverage blended with the lime cold pressed oil according to Example 15, four or more scores were obtained for all items and generation of an abnormal odor was suppressed while maintaining a fresh and preferable flavor note originally possessed by an oil derived from a citrus peel as a raw material as compared with the PET-bottled acidic beverage blended with Comparative Example 8 in which a linoleic acid reduction treatment was not performed, and therefore, a content ratio of linoleic acid with respect to a content of citral as a main aroma component was out of a specified range.

Although the present invention has been described in detail and with reference to specific embodiments, it will be apparent to those skilled in the art that various changes and modifications can be made without departing from the spirit and scope of the present invention. This application is based on a Japanese Patent Application (No. 2021-168177) filed on Oct. 13, 2021, the contents of which are incorporated herein as a reference.

1. A purified product of a citrus oil, wherein an oil derived from a citrus peel is used as a raw material,
a content of linoleic acid is 0.015 parts by mass or less with respect to 1 part by mass of citral, or 0.063 parts by mass or less with respect to 1 part by mass of linalool, or 0.083 parts by mass or less with respect to 1 part by mass of decanal, and
a content of sesquiterpene hydrocarbons is 20 mass % or less.

2. The purified product of a citrus oil according to claim 1, wherein an oil derived from a lemon peel is used as a raw material,
a content of linoleic acid is 0.015 parts by mass or less with respect to 1 part by mass of citral, and
a content of sesquiterpene hydrocarbons is 20 mass % or less.

3. The purified product of a citrus oil according to claim 1, wherein an oil derived from a lime peel is used as a raw material,
a content of linoleic acid is 0.008 parts by mass or less with respect to 1 part by mass of citral, and
a content of sesquiterpene hydrocarbons is 20 mass % or less.

4. The purified product of a citrus oil according to claim 1, wherein an oil derived from an orange peel is used as a raw material,
a content of linoleic acid is 0.063 parts by mass or less with respect to 1 part by mass of linalool, and
a content of sesquiterpene hydrocarbons is 10 mass % or less.

5. The purified product of a citrus oil according to claim 1, wherein an oil derived from a grapefruit peel is used as a raw material,
a content of linoleic acid is 0.083 parts by mass or less with respect to 1 part by mass of decanal, and
a content of sesquiterpene hydrocarbons is 10 mass % or less.

6. A fragrance or flavor composition, comprising the purified product of a citrus oil according to claim 1.

7. A beverage or food product, a fragrance or cosmetic product, a pharmaceutical product, or an oral care product, comprising the fragrance or flavor composition according to claim 6.

8. A production method of the purified product of a citrus oil according to claim 1, comprising at least one selected from the group consisting of subjecting an oil as a raw material to an aqueous alkali solution washing treatment, subjecting an oil as a raw material to an ion exchange resin treatment, and subjecting an oil as a raw material to a treatment in which thin-film distillation and liquid-liquid extraction are combined.

9. A production method of the purified product of a citrus oil according to claim 1, comprising:
subjecting an oil as a raw material to an aqueous alkali solution washing treatment, wherein
an alkali substance used in the aqueous alkali solution washing treatment is a strong base, and
when the oil as the raw material is treated without being concentrated and is subjected to the aqueous alkali solution washing treatment, an amount of the alkali substance used is 0.05 mol or less with respect to 1 kg of a treatment target.

10. A production method of the purified product of a citrus oil according to claim 1, comprising:
subjecting an oil as a raw material to an ion exchange resin treatment, wherein
an ion exchange resin used in the ion exchange resin treatment is an anion exchange resin, and
an amount of the anion exchange resin used is 0.03 kg or more with respect to 1 L of a treatment target.
